(19) United States
(12) Patent Application Publication
(10) Pub. No.: US 2026/0232564 A1
(43) Pub. Date: Aug. 13, 2026

(54) ANTI-WRINKLE SKINCARE COMPOSITION COMPRISING A STABLE HOMOGENEOUS AQUEOUS SUSPENSION OF BIOPOLYMERS

(71) Applicant: 11584022 CANADA INC., Montreal (CA)

(72) Inventors: Thomas Di Nardo, Montreal (CA); George Deckner, Cincinnati, OH (US)

(73) Assignee: 11584022 CANADA INC., Montreal, QC (CA)

(21) Appl. No.: 19/156,770

(22) PCT Filed: Dec. 29, 2023
(86) PCT No.: PCT/IB2023/063395

§ 371 Date: Aug. 14, 2025

Related U.S. Application Data
(60) Provisional application No. 63/485,000, filed on Feb. 14, 2023.

(51) Int. Cl. A61K 8/73; A61K 8/04; A61Q 19/08

(52) U.S. Cl. CPC A61K 8/736; A61K 8/042; A61K 8/731; A61Q 19/08; A61K 2800/48; A61K 2800/522; A61K 2800/524

(57) ABSTRACT

Described herein are skincare compositions with beneficial skin properties, including smoothing the appearance fine lines and/or wrinkles. The compositions are formulated for a dermatologically safe application to human skin and includes (i) biopolymer molecules that have been mechanically processed into a stable homogeneous aqueous suspension and (ii) at least one of a humectant, an emollient, an occlusive, a ceramide, an emulsifier, a thickening agent, a preservative, an antioxidant, a fragrance, a functional ingredient, a UVB filter, a UVA filter, and a broad-spectrum filter. The stable homogeneous aqueous suspension comprises insoluble and/or semi-soluble biopolymer particles stably dispersed within a polar solvent. Contrary to existing compositions, the stably dispersed biopolymer molecules of the invention have not been obtained by solubilisation into an acidic solution or by solubilisation into a basic solution.

### CROSS REFERENCE TO RELATED APPLICATION

The present application claims priority to provisional patent application U.S. 63/485,000 filed Feb. 14, 2023, the content of which is incorporated herein in its entirety.

### FIELD OF THE INVENTION

The invention relates to the field of cosmetics, and more particularly to skincare compositions with beneficial skin properties, including smoothing the appearance fine lines and/or wrinkles.

### BACKGROUND OF THE INVENTION

The cosmetic industry is permanently searching for new natural, biocompatible, biodegradable and non-toxic ingredients. The cosmetic industry is also permanently searching for composition with beneficial properties to the human skin.

Natural polymers, or biopolymers, are polymers that are abundant, natural and renewable, making them an attractive resource for the cosmetic industry. However, most abundant biopolymers such as cellulose and chitin are insoluble, thereby limiting and complicating their use. Advantageously, Applicant has described in International PCT publication WO 2022/137184 new stable and homogeneous suspensions of biopolymers that may find numerous applications in the cosmetic industry. International patent application PCT/IB2023/056723 filed Jun. 29, 2023, which also belongs to the present Applicant, describes cosmetic formulations comprising a biopolymer.

Nevertheless, there is still a need for compositions displaying beneficial skincare properties, including protection of the skin, smoothing the appearance fine lines and/or wrinkles, whitening of age spots, regenerating the skin, etc.

The present invention addresses these needs and other needs as it will be apparent from the review of the disclosure and description of the features of the invention hereinafter.

### BRIEF SUMMARY OF THE INVENTION

According to one aspect, the invention relates to a skincare composition for application to human skin, said composition comprising biopolymer molecules that have been mechanically processed into a stable homogeneous aqueous suspension, and wherein said stable homogeneous aqueous suspension comprises insoluble and/or semi-soluble biopolymer particles stably dispersed within a polar solvent.

According to one particular aspect, the invention relates to a skincare composition comprising: (i) biopolymer molecules that have been mechanically processed into a stable homogeneous aqueous suspension, and wherein said stable homogeneous aqueous suspension comprises insoluble and/or semi-soluble biopolymer particles stably dispersed within a polar solvent; and (ii) at least one of a humectant, an emollient, an occlusive, a ceramide, an emulsifier, a thickening agent, a preservative, an antioxidant, a fragrance, a functional ingredient, a UVB filter, a UVA filter, and a broad-spectrum filter; and wherein said skincare composition is formulated for a dermatologically safe application to human skin.

According to another aspect, the invention relates to a skincare composition for application to human skin, said composition comprising biopolymer insoluble and/or semi-soluble biopolymer molecules that have been mechanically processed into a stable homogeneous aqueous suspension, and wherein said skincare composition provides for at least one skincare benefit selected from the group consisting of:
i. smoothing the appearance of fine lines and/or wrinkles, and/or effective in reducing their presence;
ii. improving the appearance of bags under the eyes and facial wrinkles;
iii. protecting the skin;
iv. whitening of age spots;
v. mattifying the skin;
vi. regenerating and/or rejuvenating the skin;
vii. repairing and/or healing the skin;
viii. densifying the skin and/or improving one or more mechanical properties of the skin;
ix. improving suppleness of the skin;
x. creating a protective film on the skin, e.g. a long lasting protective film lasting at least 24 hours, or at least a week, or at least ten (10) days;
xi. creating on the skin a film almost dry to the touch;
xii. creating a cooling sensation on the skin;
xiii. hydrating and/or moisturizing the skin;
xiv. preventing water evaporation from the skin;
xv. improving skin barrier integrity;
xvi. reducing epidermal water loss and/or improving skin water retention; and
xvii. non-comedogenic.

According to another aspect, the invention relates to a method of skin protection, comprising applying onto the skin a skincare composition as defined herein.

According to another aspect, the invention relates to a method for treating skin wrinkles, comprising applying a composition as defined herein on a surface of the skin of a human subject in need thereof.

According to another aspect, the invention relates to the use of a composition as defined herein, for at least one of preventing apparition of skin wrinkles and removing skin wrinkles.

According to another aspect, the invention relates to the use of a composition as defined herein, in the manufacture of a skincare cosmetic.

According to another aspect, the invention relates to a skincare kit comprising two-part formulations that are activated when combined, wherein at least one of the two-part formulations comprises a skincare composition as defined herein.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

For the invention to be readily understood, embodiments of the invention are illustrated by way of examples in the accompanying figures.

FIG. 1: Pictures showing application scheme and patterns for the test of Example 2.

FIG. 2: Pictures showing samples in the jar which were tested in accordance with Example 2.

FIG. 3: Table displaying visual and sensory scoring of samples 1 to 3, tested on the skin in accordance with Example 2. The score is from 1 to 10, where a score of 1 is the worst and a score of 10 is the best.

FIG. 4: Table displaying visual and sensory scoring of samples 4 to 6, tested on the skin in accordance with Example 2. The score is from 1 to 10, where a score of 1 is the worst and a score of 10 is the best.

Further details of the invention and its advantages will be apparent from the detailed description included below.

### DETAILED DESCRIPTION OF EMBODIMENTS

In the following description of the embodiments, references to the accompanying figures are illustrations of an example by which the invention may be practised. It will be understood that other embodiments may be made without departing from the scope of the invention disclosed. Unless defined otherwise, all technical and scientific terms used herein have the same meaning as commonly understood by one of ordinary skill in the art to which the invention belongs.

### General Overview

In International PCT publication WO 2022/137184 (incorporated herein by reference in its entirety) Applicant describes the preparation of stable homogeneous suspensions of insoluble and/or semi-soluble biopolymers. In international patent PCT application PCT/IB2023/056723 Jun. 29, 2023 (also incorporated herein by reference in its entirety), Applicant describes cosmetic formulations comprising a biopolymer.

The present invention generally relates to the uses of such suspensions and/or formulations in the manufacture of “skincare composition(s)” or “skincare formulation(s)” (both terms are used herein interchangeably) for use on human skin. Particular aspects concern new protective and/or skincare compositions displaying beneficial skincare properties including, but not limited to, protection of the skin, smoothing the appearance fine lines and/or wrinkles, whitening of age spots, regenerating the skin, etc.

### Biopolymer

The skincare compositions in accordance with the present invention minimally require a biopolymer. As used herein, the term “biopolymer” refers to natural polymers produced by the cells of living organisms. Biopolymers consist of monomeric units that are covalently bonded to form larger molecules. The present invention encompasses polypeptides, polysaccharides and polynucleotides biopolymers. Other examples of biopolymers include natural rubbers (polymers of isoprene), suberin and lignin (complex polyphenolic polymers), cutin and cutan (complex polymers of long-chain fatty acids) and melanin. In embodiments the biopolymers used as starting materials and obtained in the suspensions are substantially pure, i.e., they consist of only purified natural polymers.

Preferably, the biopolymer used in the skincare formulation is substantially free from chemical residues and any of such chemical residue is absent or present in undetectable or trace amounts. Preferably, the biopolymer is substantially free from chemical residues and any of such chemical residue is absent or present in undetectable or trace amounts. As used herein, “substantially free from chemical residues” means that chemical compounds, such as acids, bases, reactive chemicals, organic salts and/or inorganic salts, surfactants, dispersing agents (e.g., Tween 80™), a silanizing reagent, acrylamide, etc. are totally absent or merely present in undetectable or trace amounts in the final composition or final suspension. In embodiments, the biopolymer(s) will constitute at least 98%, or at least 99% or at least 99.9% or at least 99.99% by weight of the organic compounds in the biopolymer composition or suspension, i.e., the biopolymer composition or suspension will contain less than 2% or less than 1%, less than 0.1%, or less than 0.01%, or less than 0.001% by weight of organic components other than the biopolymer(s) or degradation product(s). In embodiments, the skincare composition according to the invention comprises biopolymer molecules that have been mechanically processed into a stable homogeneous aqueous suspension, but in absence of chemical compounds such as acids, bases, or other chemicals that are typically used in the solubilization and/or mechanical processing of biopolymers. In other words, the skincare composition according to the invention comprises stably dispersed biopolymer molecules which have not been obtained by solubilisation into an acidic solution (e.g. pH<5.5; acetic acid) or by solubilisation into a basic solution (e.g., pH >8.5; NaOH). In other embodiments, the skincare composition according to the invention comprises stably dispersed biopolymer molecules which have not been solubilized because of chemical modifications such as carboxylation and/or alkylation. In accordance with the present invention, the biopolymer may be insoluble or semi-soluble in water. As used herein, the term “insoluble biopolymer” refers to a biopolymer that is “insoluble” in a polar solvent (particularly water) and this term encompasses equivalent terms such as “non-water-soluble”, or “not soluble in water”, or “water-insoluble” or “indissoluble”. Insolubility can typically be observed by a separation, i.e., two separate phases in an aqueous mixture, for instance biopolymer deposits/sediments at a bottom or floating at the top of the aqueous mixture. In accordance with the present invention, examples of insoluble biopolymers include, but are not limited to, chitin, chitosan, cellulose, hemicellulose, lignin, amylose, actin, fibrin, collagen, silk, fibroin, keratin, wool, alginic acid and mixtures thereof. As used herein, the term “semi-soluble biopolymer” refers to a biopolymer that may be solubilized in a polar solvent such as water, but under certain conditions (e.g., molecular weight, heat, addition of chemicals such as acids, alcohols, surfactants, etc.). In accordance with the present invention, examples of semi-soluble biopolymers include, but are not limited to gelatin, pectin, starch, amylopectin, agarose, hyaluronic acid, RNA, DNA, xanthan gum, latex, polymannans, suberin, cutin, cutan, and mixtures thereof.

In embodiments the insoluble biopolymer is selected from chitin, chitosan, cellulose, hemicellulose, lignin, amylose, actin, fibrin, collagen, silk, fibroin, keratin, wool, and mixtures thereof. In embodiments the semi-soluble biopolymer is selected from gelatin, pectin, starch, amylopectin, agarose, alginic acid, alginate, hyaluronic acid, RNA, DNA, xanthan gum, guar gum, carrageenan, latex, polymannans, suberin, cutin, cutan, and mixtures thereof.

Various sources of biopolymers may be used, and the present invention is not limited to particular sources of materials. For instance, suitable sources of chitin may include, but are not limited to, green plants, algae, and fungi. Suitable sources of chitin and chitosan may include, but are limited to, fungi, crustaceans (e.g., crabs and shrimps) and insects. In embodiments the insoluble or semi-soluble biopolymer is obtained from fungi and mushrooms. In embodiments the insoluble or semi-soluble biopolymer is obtained from plant materials including, but not limited to, roots, tubers, leaves, petals, seeds, fruits, etc. Preferred sources are non-animal sources.

In embodiments the biopolymer is a 100% natural biopolymer such as SunSpheres Bio™ (microcrystalline cellulose, Dow Chemical), Chemjac™ (amorphophallus konjac root extract and xanthan gum, Chemspire), Kelset™ (sodium alginate, Dupont), Instant Pure-Flo F™ (corn starch, Ingredion), Gelcarin™ GP 379 (carrageenan, iota form, Dupont), Betafib™ MCF (cellulose and water-microfibrillated cellulose, Cosun Biobased Products), Betafib™ ETD (cellulose and cellulose gum-microfibrillated cellulose, Cosun Biobased Products), Exilva™ FM02-V,L (cellulose-microfibrillated cellulose, Borregard), Naturesoft™ 800 (cellulose-micro powders), Kelcogel™ CG-HA (gellan gum-CP, Kelco), agar agar, and agarose, mushroom chitosan (e.g., GBS003, Qingdao Chibio Biotech), fungal chitosan (e.g., GBS010, Qingdao Chibio Biotech; or from Kraeber & Co), a fungal derived chitosan (e.g., Kiosmetine-CS™, Kitozyme).

In embodiments the biopolymer is a natural derived biopolymer such as Natrathix™ bio cellulose (cellulose gum, Ashland), Aquasorb™ A500 (cellulose gum, Ashland), Polysurf™ CS 67/Natrosol™ CS plus 330 (cetyl hydroxyethylcellose, Ashland), Structure XL™ (hydroxy propyl starch phosphate, Nouryon), CD-58 (chitosan succinimide, Onlystar Bio-Technology Ltd), carboxymethyl chitosan derivative (GBS010, Qingdao Chibio Biotech), Makimousse™ 7/400 (sodium polyacrylate starch-kobo products, Daito Kasei Kogyo), Salanjul/Sanfresh™ 1000/300sp (sodium polyacrylate starch, Iwase Cosfa USA Inc./Sanyo), Antaron ECoT ethylcellulose (ethyl cellulose, Ashland).

In embodiments the biopolymer comprises a synergistic biopolymer combination, such as Chemjac™ (amorphophallus konjac root extract and xanthan gum, Chemspire), PemuPur™ start (microcrystalline cellulose (and) sphingomonas ferment extract (and) cellulose gum, (Lubrizol)), Nomcort CG (xanthan gum, ceratonia siliqua gum, Ikeda).

The biopolymer may also comprise other biopolymer actives such as B-CAN™ 55% (oat beta glucan, Adams Food Ingredients) and/or mushroom derived beta glucan.

The present invention encompasses mixtures of two, three, four, five or more insoluble biopolymers including, but not limited to, chitin+chitosan, chitin+ cellulose, chitin+collagen, chitin+silk, chitosan+silk, chitosan+ cellulose, chitosan+collagen, cellulose+collagen, cellulose+silk, collagen+silk, etc. The present invention also encompasses mixtures of two, three, four, five or more semi-soluble biopolymers including, but not limited to agarose+DNA, xanthan gum+starch, latex+alginate, xanthan gum+DNA, guar gum+cutan, etc. It may also be envisioned to mix together two, three, four, five or more insoluble and semi-soluble biopolymers including but not limited to chitin+agarose, chitosan+agarose, chitin+gelatin, chitin+xanthan gum, chitosan+xanthan gum, chitin+sodium hyaluronate, chitosan+sodium hyaluronate, cellulose+sodium hyaluronate, chitin+agarose, chitosan+agarose, cellulose+agarose.

The present invention encompasses also combinations of hydrophobically modified biopolymers and unmodified biopolymers which can form stable, viscous oil in water emulsions when dispersed using high shear processes and/or mechanical energy (with or without emulsifiers), Examples of hydrophobically modified biopolymers include Natrosol™ CS Plus 330/Polysurf™ CS 67 (cetyl hydroxyethylcellose, Ashland), StarDesign Ultra™ (sodium starch octenylsuccinate, Cargill Beauty), Inutec™ SP1 (inulin lauryl carbamate, Beneo), Texturlux Stabil™ (hydrolyzed corn starch hydroxyethyl ether, Primient).

In embodiments, the biopolymer consists of a biopolymer composition comprising biopolymer molecules that have been mechanically processed into a stable homogeneous aqueous biopolymer suspension. As used herein the term “homogeneous” generally refers to the appearance of the suspension under the naked eye (e.g., uniform color, uniform texture, etc.). Homogenous as used herein does not exclude the possibility that the suspension is “heterogenous” at the molecular level (e.g., various particles size, presence of aggregates, etc.). As used herein, the terms “stable homogeneous aqueous biopolymer suspension”, or similar terms that may be used herein interchangeably such as “homogenous biopolymer suspension” or “stable biopolymer suspension” or simply “biopolymer suspension”, all refer to a suspension of insoluble and/or semi-soluble biopolymer particles that have been stably dispersed within a polar solvent. The polar solvent may be a polar protic solvent or a polar aprotic solvent. The polar solvent may be an aqueous solvent. The insoluble and/or semi-soluble biopolymer particles that are present in biopolymer suspensions may be shaped like fibers and/or like agglomerated spheres or agglomerated bodies. Stability of the biopolymer suspensions may be assessed by any suitable means. In preferred embodiments, the stability is measured or observed by a lack of separation, i.e., one single phase instead of two separate phases in an aqueous mixture, for instance absence of biopolymer deposits/sediments at a bottom or floating at the top of the aqueous mixture. Preferably, biopolymer suspensions in accordance with the present invention are stable (e.g., absence of separation) for at least 1 day, or at least 1 week, or at least one month, or at least one year or more.

Notwithstanding the above, those skilled in the art understand that insoluble and/or semi-soluble biopolymers may never become truly soluble. Instead, they become “swellable” and bind water which is why the biopolymer becomes a viscous suspension during the high-shearing conditions and/or mechanical energy to which the biopolymer(s) are submitted in accordance with the present invention. Accordingly, the present invention encompasses both, “swellable biopolymers” as well as “non-swellable biopolymers” since a non swellable polymer could be swellable using high-shearing and/or mechanical energy. As used herein, swellable biopolymers encompasses biopolymers that absorb and bind water, which results in an increase in their particle size and water dispersion viscosity.

In embodiments, the biopolymer is swellable with wet ball milling. This may include, but it is not limited to, chitin, chitosan, hemicellulose and pregelatinized corn starch.

In embodiments, the biopolymer is swellable with high shear processes other than ball milling. This may include, but it is not limited to, microcrystalline cellulose, microfibrillated cellulose, nano cellulose, hairy nanocellulose, konjac glucomannan, hydroxypropyl starch phosphate, high acyl gellan gum, gellan gum, carboxymethyl starch, carboxymethyl cellulose (low ds type), agar agar, and agarose.

It is also conceivable in accordance with the present invention to use soluble biopolymers including, but not limited to, xanthan gum, diutan gum, sodium alginate, sclerotium gum.

In embodiments the skincare composition according to invention have a pH between about 6.5 and about 8.5, or about 7 to about 8. In particular embodiments, the skincare composition comprises a chitosan suspension and is has a pH between about 7.8 and about 8.1.

In embodiments, the biopolymer molecules or particles that are part of the skincare composition have been mechanically processed into a stable homogeneous aqueous biopolymer suspension. In embodiments the mechanical processing involves high-shearing conditions and/or high mechanical energy. In embodiments the high-shearing conditions and/or high mechanical energy is obtained by a process including, but not limited to mechanical shearing, shear thinning, planetary ball milling, rolling mill, vibrating ball mill, tumbling stirred ball mill, horizontal media mill, colloid milling. As indicated hereinafter, the high-shearing conditions and/or high mechanical energy can be carried out for a duration, under parameters, under suitable conditions, etc. until a desirable change of state is obtained, e.g., change of color, a change in viscosity, a change from a slurry to a paste, ointment, cream, lotion, gel or milk, etc.

Without wishing to be bound by theory, submitting the biopolymer to high-shearing conditions and/or high mechanical energy improves biopolymer performance not seen using conventional processes, including improved rheological properties such as viscosity, shear thinning properties, and high yield value. Biopolymers dispersed using high shear processes with a fatty alcohol may contain lamellar crystalline gel networks (LGN) that may synergistically increase the viscosity of the biopolymer dispersions. Oil+water biopolymer dispersions in accordance with the present invention may also contain Pickering emulsion wherein the water-in-oil or oil-in-water emulsion is stabilized by the biopolymer.

In embodiments the high-shearing conditions and/or high mechanical energy requires using a suitable device or apparatus including, but not limited to, ball miller (e.g., planetary ball miller, rolling miller, vibrating ball miller, tumbling stirred ball miller, horizontal media mill, colloid miller, a magnetic miller), a twin-screw extruder, a high-pressure homogenizer, a blade homogenizer, a stirring homogenizer, a disperser, a rotor-stator homogenizer, a high-shear mixer, a plowshare mixer, a dynamic mixer, a plough mixer, a turbine mixer, a speed mixer, an attrition miller, a sonicator (e.g. high shear ultrasonic processes), a tissue tearor, a cell lysor, a polytron, a ribbon agitator, a microfluidizer, a high pressure homogenizer, and combinations thereof. In preferred embodiments, the present invention utilizes ball milling under wet conditions. Particular examples of ball millers include, but are not limited to, vertical planetary mill (e.g., Tencan XQM-2A™) with 100 ml capacity zirconia jars and 10 mm diameter zirconia balls, Flacktek™ speed mixer (DAC 330-11 SE) with 40 mL zirconia jar with 5 mm diameter zirconia balls or zirconia rings, 1.5 L Supermill Plus™ with 1.4-1.7 mm zirconia beads and Netzsch Labstar with 0.6-0.8 mm beads or 1.4-1.7 mm beads.

In particular embodiments, biopolymer compositions and suspensions in accordance with the present invention are obtained using a particular protocol referred herein as the “10+1 Alt method”. This method comprises milling of the biopolymer for a certain period of time (e.g., 10 min) followed by a short pause (e.g., 1 min) then milling in the opposite direction for a certain period of time (e.g., 10 min) for a total of 1 hour, or 2 hours, or 3 hours, or 5 hours, 10 hours, or 12 hours.

Advantageously, the viscosity of the compositions/suspensions can be altered by varying the high-shearing conditions and/or mechanical energy to which the biopolymer(s) are submitted. These conditions can be adjusted to obtain a stable homogeneous suspension (e.g., a stable colloidal homogeneous suspension) having a desired viscosity. Typically, providing more mechanical energy will increase the shearing and will reduce accordingly the viscosity of the end product. The biopolymer itself and/or the final compositions for drug delivery may be formulated as a paste, an ointment, a cream, a lotion, a gel or a milk.

In embodiments, the biopolymer, and/or the biopolymer compositions comprising biopolymer molecules or particles is a colloidal homogeneous biopolymer suspension. In embodiments, the colloidal homogeneous suspension comprises colloids having a range from about 1 nm to about 1 μm.

In embodiments, the biopolymer, and/or the biopolymer compositions comprising biopolymer molecules, comprises biopolymer fibers. In embodiments the fibers have of a width of about 1 nm to about 5 μm, or about 5 nm to about 5 μm, about 7 nm to about 5 μm, or about 10 nm to about 5 μm, or about 20 nm to about 5 μm, or about 25 nm to about 5 μm, or about 30 nm to about 5 μm, or about 35 nm to about 5 μm, or about 35 nm to about 3 μm. In embodiments the fibers having of a width of at least 1 nm, or at least 5 nm, or at least 10 nm, or at least 20 nm, or at least 30 nm, or at least 40 nm, or at least 50 nm, or at least 75 nm, or at least 100 nm, or at least 250 nm, or at least 500 nm, or at least 750 nm, or at least 1 μm, or at least 2 μm, or at least 3 μm, or at least 4 μm, or at least 5 μm, or wider.

In embodiments the biopolymer, and/or the biopolymer compositions comprising biopolymer molecules, comprises biopolymer fibers having a length of about 1 nm to about 200 μm, of about 10 nm to about 100 μm, or about 50 nm to about 10 μm, or about 100 nm to about 10 μm, or about 500 nm to about 10 μm, or about 750 nm to about 10 μm, or about 800 nm to about 10 μm, or about 900 nm to about 5 μm, or about 1 μm to about 10 μm, or about 1 μm to about 5 μm, or about 1 μm to about 3 μm. In embodiments the fibers have of a length of at least 1 nm, or at least 10 nm, at least 50 nm, or at least 100 nm, or at least 250 nm or at least 500 nm, or at least 750 nm, or at least 800 nm, or at least about 900 nm, or at least 1 μm, or at least 2 μm, or at least 3 μm, or at least 4 μm, or at least 5 μm, or at least 6 μm, or at least 7 μm, or at least 8 μm, or at least 9 μm, or at least 10 μm, or at least 25 μm, or at least 50 μm, or at least 75 μm, or at least 100 μm, or at least 150 μm, or at least 200 μm or longer. In embodiments, a dry particle size range may be between about 1 nm to about 1 μm, or up to 10 μm, and a wet particle size range may be between about 200 nm to about 20 μm, or up to 200 μm.

In embodiments the biopolymer, and/or the biopolymer compositions comprising biopolymer molecules, comprises biopolymer fibers having both: (i) a width greater than 20 nm (e.g., at least 25 nm, or at least 40 nm, or at least 50 nm) and a length greater than 50 nm (e.g., at least 100 nm, or at least 500 nm, or at least 1 μm, or at least 2 μm); or (ii) a width greater than 32 nm (e.g., at least 35 nm, or at least 40 nm, or least 50 nm) and a length of than 50 nm (e.g., at least 100 nm, or at least 500 nm, or at least 1 μm, or at least 2 μm); or (iii) a width greater than 20 nm (e.g., at least 25 nm, or at least 40 nm, or least 50 nm) and a length of than 500 nm (e.g., at least 600 nm, or at least 750 nm, or at least 1 μm, or at least 2 μm); or (iv) a width greater than 30 nm (e.g., at least 35 nm, or at least 40 nm, or least 50 nm) and a length of than 800 nm (e.g., at least 900 nm, or at least 1 μm, or at least 2 μm); or (v) a width greater than 8 nm (e.g., at least 10 nm, at least 25 nm, or at least 35 nm, or at least 40 nm, or least 50 nm) and a length of than 340 nm (e.g., at least 350 nm, or at least 500 nm, at least 750 nm, or at least 900 nm, or at least 1 μm, or at least 2 μm); or (vi) a width greater than 11 nm (e.g., at least 15 nm, at least 25 nm, or at least 35 nm, or at least 40 nm, or least 50 nm) and a length of than 166 nm (e.g., at least 200 nm, or at least 350 nm, or at least 500 nm, at least 750 nm, or at least 900 nm, or at least 1 μm, or at least 2 μm); or (viii) a width greater than 32 nm (e.g., at least 35 nm, or at least 40 nm, or least 50 nm) and a length greater than 800 nm (e.g., at least 900 nm, or at least 1 μm, or at least 2 μm, or at least 3 μm, or at least 4 μm, or at least 5 μm, or at least 6 μm, or at least 7 μm, or at least 8 μm, or at least 9 μm, or at least 10 μm, or at least 25 μm, or at least 50 μm, or at least 75 μm, or at least 100 μm, or at least 150 μm, or at least 200 μm or longer).

In embodiments the biopolymer, and/or the biopolymer compositions comprising biopolymer molecules, comprises biopolymer fibers wherein the average width and average length of the fibers in the composition are as defined hereinabove, e.g. an average width greater than 20 nm (e.g., at least 25 nm, or at least 40 nm, or at least 50 nm) and an average length greater than 50 nm (e.g., at least 60 nm, at least 75 nm, or at least 100 nm, or at least 500 nm, at least 750 nm, or at least 1 μm, or at least 2 μm, or at least 3 μm, or at least 4 μm, or at least 5 μm, or at least 6 μm, or at least 7 μm, or at least 8 μm, or at least 9 μm, or at least 10 μm, or at least 25 μm, or at least 50 μm, or at least 75 μm, or at least 100 μm, or at least 150 μm, or at least 200 μm or wider).

In embodiments the biopolymer suspensions and/or biopolymer compositions comprising biopolymer molecules, have a pH between about 6.5 and about 8.5. In particular embodiments, the biopolymer suspension is a chitosan suspension having a pH between about 7.8 and about 8.1.

In embodiments the biopolymer, and/or the biopolymer compositions comprising biopolymer molecules, comprises biopolymer fibers having both a crystalline region and an amorphous region. In embodiments the stable homogeneous suspension comprises biopolymer fibers having a globular shape. In embodiments the stable homogeneous suspension is comprised of mainly, or only, of suspended biopolymer nanofibrils.

Those skilled in the art are aware that particle size measurements may vary according to the measurement method and the state of the particles (e.g., particles in a wet state are typically larger than the same particles in a dry state). Typically, the particles will be in a wet or suspended stage when measured by dynamic light scattering (DLS) and in a dry stage when measured by scanning electron microscopy (SEM).

In embodiments the biopolymer, and/or the biopolymer compositions comprising biopolymer molecules, comprises agglomerated spheres of alginic acid having an average size of about 40 nm to about 80 nm, or about 45 nm to about 75 nm, as measured by scanning electron microscopy (SEM). In embodiments, the stable homogeneous suspension comprises agglomerated spheres of alginic acid having a median size of about 30 nm to about 70 nm or about 35 nm to about 65 nm, average size of about 40 nm to about 80 nm, or about 45 nm to about 75 nm, as measured by scanning electron microscopy (SEM).

In embodiments the biopolymer, and/or the biopolymer compositions comprising biopolymer molecules, comprises agglomerated spheres of cellulose having an average size of about 50 nm to about 80 nm, or about 55 nm to about 75 nm, average size of about 40 nm to about 80 nm, or about 45 nm to about 75 nm, as measured by scanning electron microscopy (SEM). In embodiments the stable homogeneous biopolymer suspension comprises agglomerated spheres of cellulose having a median size of about 35 nm to about 75 nm or about 40 nm to about 65 nm, average size of about 40 nm to about 80 nm, or about 45 nm to about 75 nm, as measured by scanning electron microscopy (SEM).

In embodiments the biopolymer, and/or the biopolymer compositions comprising biopolymer molecules, comprises agglomerated spheres of chitin having an average size of about 45 nm to about 85 nm, or about 50 nm to about 80 nm. In embodiments the stable homogeneous biopolymer suspension comprises agglomerated spheres of cellulose having a median size of about 45 nm to about 80 nm or about 50 nm to about 75 nm, as measured by scanning electron microscopy (SEM).

In embodiments the biopolymer, and/or the biopolymer compositions comprising biopolymer molecules, comprises agglomerated spheres of chitosan having an average size of about 75 nm to about 120 nm, or about 80 nm to about 115 nm, or about 85 nm to about 110 nm, as measured by scanning electron microscopy (SEM). In embodiments the stable homogeneous suspension comprises agglomerated spheres of chitosan having a median size of about 70 nm to about 100 nm or about 75 nm to about 95 nm, as measured by scanning electron microscopy (SEM).

In embodiments the biopolymer, and/or the biopolymer compositions comprising biopolymer molecules, comprises agglomerated spheres of silk having an average size of about 40 nm to about 165 nm, or about 45 nm to about 160 nm, as measured by scanning electron microscopy (SEM). In embodiments the stable homogeneous biopolymer suspension comprises agglomerated spheres of silk having a median size of about 40 nm to about 150 nm or about 45 nm to about 140, as measured by scanning electron microscopy (SEM).

In embodiments the biopolymer, and/or the biopolymer compositions comprising biopolymer molecules, comprises particles of one or more of alginic acid, cellulose, chitin, chitosan and silk, wherein the range of particle sizes, as measured by SEM is as defined in the tables and figures of WO 2022/137184.

In embodiments the biopolymer, and/or the biopolymer compositions comprising biopolymer molecules, is (are) characterized by visual properties like those depicted in the SEM images shown in the figures of WO 2022/137184.

In embodiments the biopolymer, and/or the biopolymer compositions comprising biopolymer molecules, is (are) characterized by a Fourier Transform Infrared Spectroscopy (FTIR) spectrum as depicted in the figures of WO 2022/137184.

In embodiments the biopolymer, and/or the biopolymer compositions comprising biopolymer molecules, is (are) characterized by Solid-State Nuclear Magnetic Resonance characterization (SSNMR) as depicted in the figures of WO 2022/137184.

In embodiments the biopolymer, and/or the biopolymer compositions comprising biopolymer molecules, is (are) characterized by Power X-Ray Diffraction (PXRD) pattern(s) as depicted in the figures of WO 2022/137184.

In embodiments the biopolymer, and/or the biopolymer compositions comprising biopolymer molecules, is (are) characterized by Dynamic Light Scattering (DLS) measurements like those reported in WO 2022/137184.

In embodiments the biopolymer, and/or the biopolymer compositions comprising biopolymer molecules, is (are) characterized by a transmittance spectrum as shown in the figures of WO 2022/137184.

In embodiments the biopolymer, and/or the biopolymer compositions comprising biopolymer molecules, is (are) characterized by a sweep suspension test as reported in WO 2022/137184.

In embodiments the biopolymer, and/or the biopolymer compositions comprising biopolymer molecules, is (are) characterized by a rheological behaviour as depicted in the figure(s) s of WO 2022/137184.

In embodiments the biopolymer represents about 0.3% w/w to about 10% w/w of the skin care composition (e.g., about 0.3% w/w, or about 0.5% w/w, or about 0.75% w/w, or about 1% w/w, or about 1.5% w/w, or about 2% w/w, or about 2.5% w/w, or about 3% w/w, or about 3.5% w/w, or about 4% w/w, or about 4.5% w/w, or about 5% w/w, or about 5.5% w/w, or about 6% w/w, or about 6.5% w/w, or about 7% w/w, or about 7.5% w/w, or about 8% w/w, or about 8.5% w/w, or about 9% w/w, or about 9.5% w/w, or about 10% w/w). It is also conceivable according to the present invention to reach higher concentrations of biopolymer in the skin care composition (e.g., about 11% w/w, about 12% w/w, about 13% w/w, about 14% w/w, about 15% w/w).

In embodiments the total amount of biopolymer+water in the skin care composition is closed to 100% w/w (e.g. about 99.9% w/w, or about 99.5% w/w, or about 99% w/w, about 98.5% w/w, or about 98% w/w, or about 97.5% w/w, or about 97% w/w, or about 96.5% w/w, or about 96% w/w, or about 95.5% w/w, or about 95% w/w). In embodiments the total amount of biopolymer+water in the skin care composition is equal or greater than 90% w/w (e.g., at least 90% w/w, or at least 91% w/w, or at least 92% w/w, or at least 93% w/w, or at least 94% w/w, or at least 95% w/w, or at least 95.5% w/w, or at least 96% w/w, or at least 96.5% w/w, or at least 97% w/w, or at least 97.5% w/w, or at least 98% w/w, or at least 98.5% w/w, or at least 99% w/w, or at least 99.5% w/w). In embodiments the total amount of biopolymer+water in the skin care composition is between 90% w/w and 99% w/w, or between 92% w/w and 97% w/w, or between 93% w/w and 96% w/w.

### Skincare Compositions

One particular aspect of the invention concerns skincare compositions comprising a biopolymer and/or a biopolymer suspension as described herein. In embodiments the biopolymer comprises, or alternatively consists of, a biopolymer composition comprising biopolymer molecules that have been mechanically processed into a stable homogeneous aqueous suspension (e.g., a suspension of insoluble and/or semi-soluble particles stably dispersed within a polar solvent).

In embodiments, the skincare composition comprises a biopolymer and at least one of a humectant, an emollient, an occlusive, a ceramide, an emulsifier, a thickening agent, a preservative, an antioxidant, a fragrance, a functional ingredient, a UVB filter, a UVA filter, and a broad-spectrum filter. In embodiments, the skincare composition comprises at least a biopolymer, an emulsifier/stabiliser, and a preservative.

In embodiments the humectant is selected from the group consisting of glycerin, urea, hyaluronic acid, salicylic acid, alpha hydroxy acid, propylene glycol, honey, sorbitol, butylene glycol, sodium PCA, allantoin, sodium lactate, dicyanamide, tremella extract, seaweed, aloe vera and mixtures thereof. In embodiments, the humectant represents up to about 20% w/w of the skin care composition (e.g., about 0.1% w/w, or about 0.5% w/w, or about 1% w/w, or about 2% w/w, or about 5% w/w, or about 10% w/w, or about 15% w/w, or about 20% w/w).

In embodiments the emollient is selected from the group consisting of beeswax, butylphthalimide isopropylphthalimide, candelilla oil, castor oil, cocoa butter, coconut oil, cyclomethicone, cyclopentasiloxane, dicaprylyl ether, diisopropyl dimer dilinoleate, dimethicone, dipropylene glycol dibenzoate, glycerin, hydrogenated polyisobutene, isocetyl stearoyl stearate, Isododecane, isohexadecane, isoparaffin, isopropyl isostearate, isopropyl palmitate, isopropyl stearate, jojoba oil, lanolin, mineral oil, octyl dodecanol, octyl palmitate, olive oil, palm, pentaerythritol tetraoctanoate, petrolatum, phenyl ethyl benzoate, polyglyceryl-4 oleate, propylene glycol, propylene glycol dibenzoate, propylene glycol isostearate, shea butter, sorbitol, squalane, sunflower, trisiloxane, and mixtures thereof. In embodiments, the emollient represents up to about 15% w/w of the skin care composition (e.g., about 0.1% w/w, or about 0.5% w/w, or about 1% w/w, or about 2% w/w, or about 5% w/w, or about 10% w/w, or about 15% w/w).

In embodiments the occlusive is selected from the group consisting of mineral oil, petrolatum, lanolin, dimethicone, shea butter and mixtures thereof. In embodiments, the occlusive represents up to about 10% w/w of the skin care composition (e.g., about 0.1% w/w, or about 0.5% w/w, or about 1% w/w, or about 2% w/w, or about 5% w/w, or about 10% w/w).

In embodiments the ceramide is selected from the group consisting of ceramide EOS, ceramide NS, ceramide NG, ceramide NP, ceramide AP, ceramide EOP, phytosphingosine, sphingosine and mixtures thereof. In embodiments, the ceramides represent up to about 5% w/w of the skin care composition (e.g., about 0.1% w/w, or about 0.5% w/w, or about 1% w/w, or about 2% w/w, or about 3% w/w, or about 4% w/w, or about 5% w/w).

In embodiments the emulsifier is selected from the group consisting of glyceryl stearate, sodium lauryl sulfate, sodium dioctyl sulfosuccinate, tragacanthins, lecithin, mustard seed, soy lecithin, sodium phosphates, diacetyl tartaric acid ester of monoglyceride, sodium stearoyl lactylate, and mixtures thereof. In embodiments, the emulsifier represents up to about 10% w/w of the skin care composition (e.g., about 0.1% w/w, or about 0.2% w/w, or about 0.5% w/w, or about 1% w/w, or about 2% w/w, or about 5% w/w, or about 10% w/w).

In embodiments the thickening agent is selected from the group consisting of magnesium aluminium, silicate, silica, bentonite, carbomer, cetyl palmitate, ammonium acryloyldimethyltaurate and mixtures thereof. In embodiments, the thickening agent represents up to about 5% w/w of the skin care composition (e.g., about 0.1% w/w, or about 0.5% w/w, or about 1% w/w, or about 2% w/w, or about 3% w/w, or about 4% w/w, or about 5% w/w).

In embodiments the preservative(s) is (are) selected from: pentylene glycol (e.g. Dermosoft® Pentiol Eco), 1,2-Alkanediol (e.g., Hydrolite® 6), phenoxyethanol, benzyl alcohol, sodium benzoate, potassium sorbate, ethylhexylglycerin, DMDM hydantoin, quaternium-15, imidazolidinyl urea, diazolidinyl urea, polyoxymethylene urea, sodium hydroxymethylglycinate, 2-bromo-2-nitropropane-1,3-diol (bromopol), glyoxal, parabens such as ethylparaben, butylparaben, methylparaben, propylparaben, isobutylparaben, isopropylparaben, benzylate, benzoic acid, benzyl ester, methylisothiazolinone (MIT; 2-methyl-4-isothiazoline-3-one), methylchloroisothiazolinone (CMIT; 5-chloro-2-methyl-4-isothiazolin-3-one), phenoxyethanol, 2-phenoxyethanol, Neolone 950 ™, OriStar™ MIT, Microcare™ MT, Methylchloroisothiazolinone (CMIT), phenoxyethanol, 2-phenoxyethanol, Euxyl K™400 (mixture of phenoxyethanol and 1,2-dibromo-2,4-dicyanobutane), triclosan (TSC), triclocarban (TCC), benzyl alcohol, benzalkonium chloride, citric acid, dehydroacetic acid, essential oils, grapefruit seed extract, lactic acid, levulinic acid, potassium sorbate, sodium dehydroacetate, sodium metabisulfite, sodium salicylate, sorbic acid, vitamin E, zinc pyrithione, Geogard™ ECT, Leucidal™ SF complete, Leucidal™ liquid complete, Leucidal™ SF Max, AMTicide™ coconut, phytocide aspen bark powder, NeoDefend™, GeoGard™ Ultra, MicroGuard™ arborcide OC, phytocide elderberry OS, ascorbic acid, vitamin E, beeswax, honey, essential oils (e.g., lavender, clove, rosemary, tea tree, sage, rosewood, thyme, cinnamon, neem, grape seed, citrus), grapefruit seed extract (GSE), bentonite clay, copper salts, butylated hydroxyanisole (BHA), butylated hydroxytoluene (BHT), benzoic acid, sodium benzoate, benzyl alcohol relatives, propionic acid, propionic salts, salicylic acid and derivatives, sorbic acid, calcium sorbate, potassium sorbate, bromopol, 2-bromo-2-nitropropane-1,3-diol, diazolidinyl urea, DMDM hydantoin, glyoxal, imidazolidinyl urea, polyoxymethylene urea, quaternium-15, sodium hydroxymethylglycinate, zinc pyrithione, sodium lauryl sulphate, propylene glycol, caprylyl glycol, AE Prerse PX 82™, phytoside blackcurrant powder, Lexgard Natural™, Sensiva Go Natural™, Spectrastat™, raspberry keton, ChemPhyte™, Hydrolite 6™, hexanediol, and mixtures thereof. Particular examples include, but are not limited to, hexanediol 1.5% w/w with benzyl alcohol, and phenoxyethanol at 0.5% w/w. It is within the skills of those in the art to identify and test suitable preservatives in accordance with the present invention. In embodiments, the preservative represents up to about 5% w/w of the skin care composition (e.g., about 0.1% w/w, or about 0.2% w/w, or about 0.3% w/w, or about 0.4% w/w, or about 0.5% w/w, or about 0.6% w/w, or about 0.7% w/w, or about 0.8% w/w, or about 0.9% w/w, or about 1% w/w, or about 1.5% w/w, or about 2% w/w, or about 2.3% w/w, or about 3% w/w, or about 4% w/w, or about 4.5% w/w, or about 5% w/w).

In embodiments the antioxidant is selected from the group consisting of vitamin A, vitamin C, vitamin E, vitamin B3, green tea extract, resveratrol, curcumin, lycopene, coenzyme Q10, polyphenols, C. asiatica extract, tamanu oil, and mixtures thereof. In embodiments, the antioxidant represents up to about 0.5% w/w of the skin care composition (e.g., about 0.01% w/w, or about 0.05% w/w, or about 0.1% w/w, or about 0.2% w/w, or about 0.3% w/w, or about 0.4% w/w, or about 0.5% w/w).

In embodiments the fragrance is selected from the group consisting of linalool, citronellol, limonene, geraniol, eugenol, lavender oil (Lavandula angustifolia), rose flower extract (Rosa damascene, canina, centifolia, gallica, multiflora), bergamot oil (Citrus bergamia), ylang-ylang oil (Canaga odorata), lemon (Citrus limon), lime (Citrus aurantifolia or Citrus medica), orange (Citrus sinensis), tangerine (Citrus tangerine), peppermint (Mentha piperita), spearmint (Mentha spicata), eucalyptus, cinnamon (Cinnamomum), cinnamal and mixtures thereof. In embodiments, the antioxidant represents up to about 0.2% w/w of the skin care composition (e.g., about 0.01% w/w, or about 0.25% w/w, or about 0.05% w/w, or about 0.1% w/w).

In embodiments the functional ingredient is selected from the group consisting of biological extracts, vitamins, amino acids, peptides, proteins, mycosporines, Mycosporine-like Amino Acids (MAAs), imino compounds that absorb ultraviolet radiations such as those described in patent U.S. Pat. No. 9,487,474, essential fatty acids (e.g., alpha hydroxy acids, bets hydroxy acids, beta-glucans and mixtures thereof. In embodiments, the functional ingredient represents up to about 10% w/w of the skin care composition (e.g., about 0.1% w/w, or about 0.5% w/w, or about 1% w/w, or about 2% w/w, or about 5% w/w, or about 10% w/w).

In embodiments the composition may also provide sunscreen protection and further comprises a UVB filter, a UVA filter and/or a broad-spectrum filter providing for a Sun Protection Factor (SPF) of at least 15, or at least 30, or at least 45, at least 50, or at least 60. In embodiments, the filter (i.e. any one or two or three of the UVB filter, UVA filter and broad-spectrum filter) represents up to about 20% w/w of the skin care composition (e.g., about 0.1% w/w, or about 0.5% w/w, or about 1% w/w, or about 2% w/w, or about 5% w/w, or about 10% w/w, or about 15% w/w, or about 20% w/w).

In embodiments the UVB filter is selected from the group consisting of Uvivul™ T 150 (Ethylhexyl Triazone), titanium dioxide, benzophenone-3 (oxybenzone), octinoxate (ethylhexyl methoxycinnamate), octocrylene, ethylhexyl salicylate (octisalicylate), homosalate (homomenthyl salicylate), ensulizole (phenylbenzimidazole sulfonic acid), amiloxate (isoamyl p-methoxycinnamate), polysilicone-15, diethylhexyl butamido triazone (iscotrizinol), 4-methylbenzylidene camphor (enxacamene) and mixtures thereof.

In embodiments the UVA filter is selected from the group consisting of butyl methoxydibenzoylmethane, butyl methoxydibenzoylmethane (Avobenzone™), diethylamino hydroxybenzoyl hexyl benzoate (Univul™ a plus), terephthalylidene dicamphor sulfonic acid (Mexoryl™ SX), disodium phenyl dibenzimidazole tetrasulfonate (Neo Heliopan™ ap), Tinosorb™ S (bis-ethylhexyloxyphenol methoxyphenyl triazine), Tinosorb™ M (methylene bis-benzotriazolyl tetramethylbutylphenol (and) aqua (and) decyl glucoside (and) propylene glycol (and) xanthan gum), Tinosorb™ A2B (tris-biphenyl triazine (and) aqua (and) decyl glucoside (and) butylene glycol (and) disodium phosphate (and) xanthan gum) and mixtures thereof.

In embodiments the broad-spectrum filter is selected from the group consisting of bis-ethylhexyloxyphenol methoxyphenyl triazine, tris-biphenyl triazine (nano), methylene bis-benzotriazolyl tetramethylbutylphenol, drometrizole trisiloxane, zinc oxide and mixtures thereof.

The skincare compositions in accordance with the present invention may find numerous applications, at least for benefiting the skin. For instance, the skincare compositions may provide one or more of the following benefits: moisturizing the skin, improving skin appearance, protecting from environmental damages, fighting the effects of aging, removing wrinkles and/or removing sunspots.

In embodiments the skincare composition comprises the following ingredients:

### Composition A:

| | % (w/w) IN THE |
|---|---|
| FUNCTION(S) | FORMULATION |
| biopolymer | 0.3-10 |
| (dry weight) | |
| humectant | 0-20 |
| emollient | 1-15 |
| occlusive | 0-10 |
| ceramide | 0-5 |
| emulsifier | 0.2-10 |
| thickening agent | 0-5 |
| preservative | 0.5-5 |
| antioxidant | 0-0.5 |
| fragrance | 0-0.2 |
| functional ingredient | 0-10 |
| UVB filter | 0-20 |
| UVA filter | 0-20 |
| broad-spectrum filter | 0-20 |
| Water | 30-95 |
| (to complete to 100%) | |

### Composition B:

| | % (w/w) IN THE |
|---|---|
| FUNCTION(S) | FORMULATION |
| biopolymer | 5-10 |
| (dry weight) | |
| preservative | 0.2-5 |
| Water to make 100% | 85-94.8 |

### Composition C:

| | % (w/w) IN THE |
|---|---|
| FUNCTION(S) | FORMULATION |
| biopolymer | 5-10 |
| (dry weight) | |
| stabilizer | 0.2-5 |
| Water to make 100% | 88-94.8 |

### Composition D:

| | % (w/w) IN THE |
|---|---|
| FUNCTION(S) | FORMULATION |
| biopolymer | 5-10 |
| (dry weight) | |
| emollient | 0.2-1 |
| preservative | 0.3-5 |
| stabilizer | 0.2-1 |
| Water to make 100% | 83-94.3 |

In embodiments the skincare composition is accordance with one of the following particular compositions:

### Composition 3:

| | % (w/w) IN THE |
|---|---|
| Ingredient | FORMULATION |
| chitosan | ~7.2 |
| (dry weight) | |
| water | ~92.4 |
| sodium benzoate and | ~0.4 |
| potassium sorbate | |

### Composition 1:

| | % (w/w) IN THE |
|---|---|
| Ingredient | FORMULATION |
| chitosan | ~7.2 |
| (dry weight) | |
| water | ~91.8 |
| glyceryl stearate | ~0.3 |
| cetyl alcohol | ~0.3 |
| sodium benzoate and | ~0.4 |
| potassium sorbate | |

### Composition 4:

| | % (w/w) IN THE |
|---|---|
| Ingredient | FORMULATION |
| chitosan | ~7.1 |
| (dry weight) | |
| water | ~91.5 |
| glyceryl stearate | ~0.3 |
| cetyl alcohol | ~0.3 |
| beeswax | ~0.3 |
| sodium benzoate and | ~0.4 |
| potassium sorbate | |

### Composition 2:

| | % (w/w) IN THE |
|---|---|
| Ingredient | FORMULATION |
| chitosan | ~7.1 |
| (dry weight) | |
| water | ~91.8 |
| glyceryl stearate | ~0.4 |
| cetyl alcohol | ~0.3 |
| beeswax | ~0.7 |
| sodium benzoate and | ~0.4 |
| potassium sorbate | |

### Composition 6:

| | % (w/w) IN THE |
|---|---|
| Ingredient | FORMULATION |
| chitosan | ~7.0 |
| (dry weight) | |
| water | ~90.5 |
| glyceryl stearate | ~0.5 |
| cetyl alcohol | ~0.5 |
| beeswax | ~1.1 |
| sodium benzoate and | ~0.4 |
| potassium sorbate | |

### Composition 5:

| | % (w/w) IN THE |
|---|---|
| Ingredient | FORMULATION |
| Cellulose | 5 |
| (dry weight) | |
| water | 95 |

### Composition 7:

| | % (w/w) IN THE |
|---|---|
| Ingredient | FORMULATION |
| Cellulose | 10 |
| (dry weight) | |
| water | 90 |

### Composition 8:

| | % (w/w) IN THE |
|---|---|
| Ingredient | FORMULATION |
| chitosan | ~5.7 |
| (dry weight) | |
| alpha cellulose | ~0.43 |
| preservative | ~5.0 |
| cetyl alcohol | ~0.55 |
| beeswax | ~0.93 |
| water to make 100% | ~87.4 |

### Composition 9:

| | % (w/w) IN THE |
|---|---|
| Ingredient | FORMULATION |
| chitosan | ~5.0 |
| (dry weight) | |
| alpha cellulose | ~0.4 |
| preservative | ~5.0 |
| water to make 100% | ~89.6 |

Methods of use may comprise simply applying the composition onto the skin. In embodiments the composition is applied on areas including, but not limited to, the hands, breast, neck, arms, legs, eye contour, forehead, face, and any other place on the body, as needed. Application(s) may be repeated as needed, for instance at different time interval (e.g., every hour, or every 2 h, or every 6 hour, or every 12 hours, or once a day, or twice a day, or thrice a day, or once or twice or thrice a week, etc.). Although the skincare compositions of the invention are mainly directed for human uses, those skilled in the art readily understand they could also find many suitable applications in animals.

### Desirable Properties

The skincare compositions in accordance with the present invention are preferably stable. Stability at least means that the biopolymer (e.g., fibers, spherical bodies) or any other component of the compositions, does not settle at the bottom. In embodiments the insoluble and/or semi-soluble biopolymer(s) remains in suspension for at least 1 week, or at least 1 month, or at least 6 months, or at least 12 months, or at least 18 months, or at least two years, or at least three years or more. It is within the skills of those in the art to address any phase separation displayed by skincare compositions in accordance with the present invention.

The skincare compositions in accordance with the present invention may be formulated to have a desired viscosity (e.g., viscosity of what is generally referred to as a paste, an ointment, a cream, a lotion, a gel or a milk). In embodiments the viscosity of the compositions/suspensions can be altered by varying the high-shearing conditions and/or mechanical energy to which the biopolymer(s) are submitted. In embodiments the stable homogeneous suspension comprises a viscosity of about 25 mPa to about 85 000 mPa. Table 1 below provides non-limiting examples of desirable viscosity for the compositions/suspensions in accordance with the present invention.

TABLE 1
Examples of desired viscosities

| Desired Consistency | Exemplary viscosity (mPa · s) |
|---|---|
| paste | about 40 000 to about 300 000 |
| ointment | about 20 000 to about 50 000 |
| cream | about 1 500 to about 30 000 |
| lotion | about 800 to about 4 000 |
| gel | about 1 000 to about 40 000 |
| milk | about 20 to about 2000 |

In embodiments, the viscosity the skincare compositions in accordance with the present invention may be varied by selecting accordingly the sensory enhancer, film former, emulsifier, emollients, UVB filter, UVA filter, and/or broad-spectrum filter.

The viscosity the skincare compositions in accordance with the invention may also be varied in accordance with the ratios or concentration of each of the compound entering into the composition, in addition to the biopolymer. In embodiments the weight ratio of biopolymers: other components (e.g., sensory enhancer, film former, emulsifier, emollients, UVB filter, UVA filter, and/or broad-spectrum filter, etc.) is of about 0.1:20 to about 10:20, or about 0.5:20 to about 3:20, or about 0.75:20, or about 1.0:20, 1.25:20. or about 1.5:20. In embodiments the skincare compositions in accordance with the invention comprises about 0.01-10% w/w of biopolymer, or 0.01-5% w/w of biopolymer, or 0.01-2% w/w of biopolymer, or 0.01-1% w/w of biopolymer. In embodiments the skincare compositions in accordance with the invention comprises about 0.01% w/w of biopolymer, or about 0.05% w/w of biopolymer, or about 0.1% w/w of biopolymer, or about 0.25% w/w of biopolymer, or about 0.5% w/w of biopolymer, or about 0.75% w/w of biopolymer, or about 1% w/w of biopolymer, or about 1.5% w/w of biopolymer, or about 2.5% w/w of biopolymer, or about 5% w/w of biopolymer.

The skincare compositions in accordance with the invention may also be formulated as stable emulsions comprising fatty acids (e.g., C₁₀-C₂₂ fatty acids), an oil and/or a wax, and/or comprising N-Acetyl Glucosamine, and/or emulsifiers and preservatives, and/or additives.

A list of possible additives includes, but is not limited to, preservatives, stabilizers and emulsifiers (e.g., Cetyl alcohol, Glyceryl stearate, Soy butter, PC90, Tara Gum, PSC3, PEG, Guar, Xanthan gum, Agarose, Sodium Hyaluronate, Tween 80™ Glycerol (humectant)), thickeners, dyes, powders (e.g., mica, pigment, chalk), inks, colorants, fragrances, essential oils, extracts (e.g., plant extract(s) such as aloe vera), vitamins (e.g., ascorbic acid), acids (e.g., acetic acid, citric acid, stearic acid), oils (cocoa butter, emu oil, olive oil, shea butter, silicone oil, mineral oil), metal oxides (e.g., zinc oxides), salts (e.g., sea salts, sodium lactate), honey, clay, propenyl glycol, polyethylene glycol, dry ingredients (e.g., rose petal powder, orange peel powder, chamomile flowers, calendula petals, etc.), allantoin, acetylglucosamine (GlcNAc), waxes (e.g., beeswax), peptides and proteins, pharmaceutical compounds (e.g., N-Acetyl Glucosamine, lidocaine, capsaicin, baclofen, ketamine, methylsulfonylmethane, orphenadrine, tetracaine, amitriptyline, bupivacaine, cyclobenzaprine, doxepin, gabapentin, guaifenesin, acetaminophen, ibuprofen, naproxen, diclofenac, meloxicam, piroxicam, ketoprofen, any NSAIDs), sugars (e.g. glucose, fructose, galactose, etc.), monomers of any of cellulose, starch, chitin, chitosan, alginic acid, collagen, silk, etc. The additive(s) may be added prior, during and/or after the step of high-shearing conditions and/or high mechanical energy.

In embodiments, the stabilizer is selected from the following stabilizers: agar, sodium alginate, carrageenan, guar, konjac, tragacanth, locust bean gum, psyllium, tara gum, fenugreek gum, xanthan gum, abietic acid, acetyl mannosylerythritol lipid, acrylamide/sodium acryloyldimethyltaurate copolymer, acrylates/aminoacrylates/C10-30 alkyl peg-20 itaconate copolymer, acrylates/C10-30 alkyl acrylate crosspolymer, acrylates/C5-8 alkyl acrylate copolymer, acrylates/stearyl methacrylate copolymer, acrylates/vinyl isodecanoate crosspolymer, acrylates/vinyl neodecanoate crosspolymer, acrylic acid/stearyl acrylate copolymer, acrylic acid/stearyl methacrylate/dimethicone methacrylate copolymer, bis-acryloyl poloxamer, alcaligenes polysaccharides, alcohols C9-11, allyl methacrylates crosspolymer, sweet almond oil polyglyceryl-4 esters, aluminum behenate, aluminum caprylate, aluminum dicetyl phosphate, aluminum dilinoleate, aluminum dimyristate, aluminum distearate, aluminum isostearate, aluminum isostearates/laurates/palmitates, aluminum isostearates/laurates/stearates, aluminum isostearates/myristates, aluminum isostearates/palmitates, aluminum isostearates/stearates, aluminum lanolate, aluminum monostearate, aluminum myristate, aluminum myristates/palmitates, aluminum/magnesium hydroxide stearate, ammonium acryloyldimethyltaurate/steareth-25 methacrylate crosspolymer, ammonium acryloyldimethyltaurate/steareth-8 methacrylate copolymer, ammonium acryloyldimethyltaurate/vinyl formamide copolymer, ammonium alginate, ammonium phosphatidyl rapeseedate, ammonium polyacryloyldimethyl taurate, ammonium shellacate, amodimethicone glycerocarbamate, AMP-C8-18 perfluoroalkylethyl phosphate, aphanothece sacrum polysaccharide, arachidyl alcohol, astragalus gummifer gum, astragalus gummifer root extract, avocadamide DEA, babassu acid, crosslinked bacillus/glucose/sodium glutamate ferment, dextro, laevo-batyl alcohol, hydrolyzed beeswax, synthetic beeswax, behenyl alcohol, bentonite, benzalkonium montmorillonite, benzalkonium sepiolite, bittern emulsion stabilising, brassica alcohol emollients, brassicyl isoleucinate esylate, butendiol/vinyl alcohol copolymer, butoxyhydroxypropyl cetyl hydroxyethylcellulose, butter decyl esters, butyl acrylate/isopropylacrylamide/peg-18 dimethacrylate crosspolymer, butyl babassuate, butylene glycol cocoate, butylene glycol isostearates, C1-5 alkyl galactomannan, C12-13 alcohols, C12-14 sec-pareth-3, C12-14 sec-pareth-5, C12-14 sec-pareth-7, C12-14 sec-pareth-8, C12-14 sec-pareth-9, C12-14 sec-pareth-12, C12-14 sec-pareth-15, C12-14 sec-pareth-20, C12-14 sec-pareth-30, C12-14 sec-pareth-40, C12-14 sec-pareth-50, C12-15 alcohols, C12-16 alkyl peg-2 hydroxypropyl hydroxyethyl ethylcellulose, C12-18 alkyl glucoside, Hydrogenated C12-18 triglycerides, C14-15 alcohols, C14-18 glycol, C14-22 alcohols, C15-18 glycol, bis-C16-18 alkyl glyceryl undecyl dimethicone, C18-22 alkyl peg-25 methacrylate/diethylaminoethyl methacrylate copolymer, C18-30 glycol, C18-38 alkyl hydroxystearoyl stearate, C20-22 alcohols, C20-30 glycol, C20-40 alcohols, C20-40 alkyl crylene, C22-24 pareth-33, bis-C24-28 hydroxyalkyl olivoyl glutamate, C28-52 olefin/undecylenic acid copolymer, C30-50 alcohols, calcium carboxymethyl cellulose, calcium carrageenan, calcium laurate, calcium myristate, calcium polyglutamate crosspolymer, calcium potassium carbomer, calcium saccharate, calcium starch octenylsuccinate, calcium stearate, callitris quadrivalvis resin, candelilla cera, candelilla wax, candelilla/jojoba/rice bran polyglyceryl-3 esters, Cannabis sativa seed oil glycereth-8 esters, caprylyl dimethicone ethoxy glucoside, caprylyl/capryl wheat bran/straw glycosides, carbomer 934, carboxymethyl cellulose acetate butyrate, carboxymethyl hydroxyethyl cellulose, carboxymethyl hydroxypropyl guar, carnauba wax, carthamus tinctorius oleosomes, hydrogenated castor oil behenyl esters, castor oil phosphate, hydrogenated castor oil stearyl esters, hydrogenated castor oil/sebacic acid copolymer caprate/caprylate, cellulose acetate propionate carboxylate, hydrolyzed cellulose gum, cellulose microcrystalline, ceramide NS/peg-8/succinic acid copolymer, ceratonia siliqua gum, ceresin, ceteareth-6 olivate, cetostearyl alcohol, cetyl alcohol, cetyl dimethicone peg-7 acetate, cetyl dodecenylsuccinate, cetyl hydroxyethyl cellulose, cetyl peg/ppg-7/3 dimethicone, bis-cetyl/peg-8 cetyl peg-8 dimethicone, chitosan lauramide succinimide, chitosan lauroyl glycinate, cholesterol/hdi/pullulan copolymer, citrus aurantium dulcis peel extract, citrus aurantium sinensis fiber, cocamide, cocamide DEA, cocamide MEA, cocamide MIPA, cocamidopropyl lauryl ether, cocoa butter glyceryl esters, coconut alcohol, coconut oil methylpropanediol esters, hydrolyzed corn starch hydroxyethyl ether, Cyamopsis tetragonoloba gum, decyl castorate, decyl glucoside, decyl hempseedate, 7-dehydrocholesterol, dehydroxanthan gum, dicapryl sodium sulfosuccinate, diethylene glycol/hydrogenated dimer dilinoleic acid copolymer, digalactosyl glyceryl linoleate/palmitate/oleate, diglycerin/dilinoleic acid/hydroxystearic acid copolymer, dihydrolanosterol, dihydroxyethyl cocamine oxide, dihydroxyethyl lauramine oxide, diisotridecyl lauroyl glutamate, dilauryl maleate/C20 olefin copolymer, dimaltosyl cyclodextrin, hydrogenated dimer dilinoleyl/dimethylcarbonate copolymer, dimethicone crosspolymer, dimethicone ethoxy glucoside, dimethicone/lauryl dimethicone/bis-vinyldimethicone crosspolymer, dimethicone/peg-15 crosspolymer, dimethyl capramide, dimethyl cocamine, dimethyl lauramine isostearate, dioleyl phosphate, dipropylene glycol isobornyl ether, dodecylhexadecanol, bis-ethoxydiglycol cyclohexane 1,4-dicarboxylate, ethyl hydroxyethyl cellulose, bis-ethyl ppg-behenate dimonium methosulfate, bis-(ethyl ppg-3 behenate) dimonium methosulfate, ethylene vinyl acetate copolymer, ethylene/acrylic acid copolymer, ethylene/sodium acrylate copolymer, feruloyl soy glycerides, perfluorocyclohexylmethanol, perfluoroheptane, perfluoromethylcyclohexane, perfluoromethyldecalin, ghatti gum, glucose pentaacetate, alpha-dextro-glucose pentaacetate, glycereth-7 malate, glycereth-8 hydroxystearate, glycereth-7 benzoate, hydrogenated glyceryl abietate, glycol cetearate, acetylated glycol stearate, glycosyl trehalose, grape seed oil glycereth-8 esters, grape seed oil polyglycerin-6 esters, maleated hexene/propylene copolymer, hydroxquinoline sulfate, hydroxyapatite, hydroxybutyl methyl cellulose, bis-hydroxyethoxypropyl dimethicone beeswax esters, bis-hydroxyethoxypropyl dimethicone isostearate, hydroxyethyl acrylate/sodium acryloyldimethyl taurate copolymer, hydroxyethyl cellulose, hydroxyethyl isostearyloxy isopropanolamine, hydroxypropyl cellulose, hydroxypropyl guar gum, hydroxypropyl methyl cellulose, hydroxypropyl xanthan gum, hydroxypropyltrimonium inulin, hydroxypropyltrimonium xanthan gum, hydroxystearic/linolenic/linoleic polyglycerides, hydroxystearic/linolenic/oleic polyglycerides, inulin lauryl carbamate, synthetic japan wax, jojoba oil glycereth-8 esters, hydrogenated lanolin alcohol, lanolinamide DEA, lauryl alcohol, lauryl alcohol diphosphonic acid, lauryl dodecenylsuccinate, lauryl/myristyl wheat bran/straw glycosides, hydrogenated lime seed oil, magnesium alginate, maltitol laurate, maltodextrin, methoxy peg-22/dodecyl glycol copolymer, methoxy peg/ppg-25/4 dimethicone, methyl cellulose, methyl vinyl ether-maleic anhydride copolymer, montmorillonite, myrist/palmitamidobutyl guanidine acetate, myristyl alaninate, myristyl alcohol, oleic/linoleic/linolenic polyglycerides, olive alcohol, hydrogenated olive oil caprylyl esters, hydrogenated olive oil cetyl esters, hydrogenated olive oil decyl esters, hydrogenated olive oil hexyl esters, hydrogenated olive oil lauryl esters, hydrogenated olive oil myristyl esters, hydrogenated olive oil stearyl esters, hydrogenated orange seed oil, ozokerite, palm kernel amide DEA, palm kernel amide MEA, palm kernelamide MIPA, palmamide DEA, palmamide MEA, palmamide MIPA, peanutamide MEA, peanutamide MIPA, pectin, tris(peg-2 phenylalanylcarboxamido)cyclohexane, peg-2 tallowamide DEA, peg-4 peg-12 dimethicone, peg-5 pentaerythrityl dimethylol propionate-2 dendrimer, peg-5 pentaerythrityl dimethylol propionate-3 dendrimer, peg-5 pentaerythrityl dimethylol propionate-4 dendrimer, peg-7 propylheptyl ether, peg-7m, peg-8 dimethicone/polysorbate 20 crosspolymer, peg-8 propylheptyl ether, peg-9m, peg-12 carnauba, peg-12 glyceryl linoleate, peg-14m, peg-20m, peg-23m, peg-65m, peg-90m, peg-100/IPDI copolymer, peg-114 polylactic acid, peg-115m, peg-160m, peg-180m, peg-400, peg-45/dodecyl glycol copolymer, peg-450, peg-500, peg/ppg-10/3 oleyl ether dimethicone, peg/ppg-100/70 tocopheryl ether, bis-peg/ppg-15/5 dimethicone, peg/ppg-18/18 isostearate, peg/ppg-18/18 laurate, peg/ppg-2/5 tocopheryl ether, peg/ppg-20/23 dimethicone, bis-peg/ppg-20/5 peg/ppg-20/5 dimethicone, peg/ppg-2000/200 copolymer, peg/ppg-23/6 dimethicone, peg/ppg-30/10 tocopheryl ether, peg/ppg-5/10 tocopheryl ether, peg/ppg-5/20 tocopheryl ether, peg/ppg-5/30 tocopheryl ether, peg/ppg-50/20 tocopheryl ether, peg/ppg-6/4 dimethicone, peg/ppg-70/30 tocopheryl ether, peg/ppg-8/3 laurate, pentadecyl alcohol, petrolatum wax microcrystalline, phosphatidic acid, phosphatidyl serine, phosphatidylglycerol, pineamidopropyl betaine, poly C10-30 alkyl acrylate, polyacrylate crosspolymer-4, polyacrylate crosspolymer-6, polyacrylate crosspolymer-11, polyacrylate crosspolymer-14, polyacrylate-10, polyacrylate-11, polyacrylate-27, polyacrylate-28, polyacrylic acid, polyester-14, polyester-15, polyethylene/isopropyl maleate/MA copolyol, polyglyceryl-2 diisostearate/ipdi copolymer, polyglyceryl-3 sunflowerseedate/citrate crosspolymer, polyglyceryl-4 diisostearate/polyhydroxystearate/sebacate, polyglyceryl-6 behenate, polypropanediol, polypropylene terephthalate, polyquaternium crosspolymer-2, polyquaternium-65, polyquaternium-83, polyquaternium-102, polyquaternium-103, polysilicone-25, polyurethane-29, polyvinyl acetate, polyvinyl pyrrolidone, potassium alginate, potassium behenoyl hydrolyzed rice protein, potassium behenoyl hydroxyproline, potassium carbomer, potassium carrageenan, potassium stearoyl hydrolyzed rice protein, potassium undecylenoyl alginate, potassium undecylenoyl carrageenan, potassium undecylenoyl hydrolyzed corn protein, potassium undecylenoyl hydrolyzed soy protein, potassium undecylenoyl hydrolyzed wheat protein, hydrolyzed potato tuber extract, ppg-4 jojoba alcohol, ppg-4 laureth-2, ppg-4 laureth-5, ppg-6-laureth-3, ppg-20 tocophereth-5, ppg-10 jojoba acid, ppg-2-buteth-2, propyl ester of PVM/MA copolymer, prunus amygdalus dulcis oil unsaponifiables, pseudozyma epicola/Camellia sinensis seed oil/glucose/Glycine soja meal/malt extract/yeast extract ferment filtrate, PVP montmorillonite, PVP/decene copolymer, pyrus malus fiber, quaternium-90 sepiolite, rhamnolipids, sclerotium gum, hydrogenated sesame seed oil, sesquiethoxytriethanolamine, sesquioctyldodecyl lauroyl glutamate, shea butter glycerides, silica dimethyl silylate, silica silylate, beta-sitosterol, sodium acrylate/acryloyldimethyltaurate/dimethylacrylamide crosspolymer, sodium acrylate/sodium acryloyldimethyl taurate copolymer, sodium acrylate/sodium acryloyldimethyl taurate/acrylamide copolymer, sodium acrylate/vinyl alcohol copolymer, sodium acrylates/vinyl isodecanoate crosspolymer, sodium acryloyldimethyl taurate/acrylamide/VP copolymer, sodium acryloyldimethyltaurate/VP crosspolymer, sodium arachidate, sodium C4-12 olefin/maleic acid copolymer, sodium carbomer, sodium carboxymethyl cellulose, sodium carboxymethyl dextran, sodium carboxymethyl starch, sodium carrageenan, sodium cellulose sulfate binding, sodium cocoyl barley amino acids, sodium cocoyl/stearoyl (alanine/arginine/asparagine/aspartic acid/glutamic acid/glutamine/glycine/histidine, sodium cyclodextrin sulfate, sodium dextrin octenylsuccinate, sodium laneth sulfate, sodium polyacrylate, sodium polyacrylate starch, sodium polyacryloyldimethyl taurate, sodium polygamma-glutamate, sodium polygamma-glutamate crosspolymer, sodium polyglutamate crosspolymer, sodium polymethacrylate, sodium polynaphthalenesulfonate, sodium polystyrene sulfonate, sodium starch octenyl succinate, sodium styrene/MA copolymer, sodium tocopheryl phosphate antioxidants, sodium trehalose octenylsuccinate, sodium/TEA-undecylenoyl alginate, sodium/TEA-undecylenoyl carrageenan, sorbitan palmate, soy protein phthalate, soyamide DEA, Sparassis crispa extract, starch hydroxypropyltrimonium chloride, iso-steareth-200 palmitate, stearic acid, stearyl alcohol, stearyl glycol, stearyl vinyl ether/MA copolymer, Sterculia urens gum, stigmasteryl chloride, stigmasteryl nonanoate, stigmasteryl succinate, styrene/ma copolymer, sucrose polypalmate, sunflower seed oil ethyl ferulate esters, sunflower seed oil polyglyceryl-10 esters, sunflower seed oil polyglyceryl-6 esters, tallow alcohol, tallow amide cosmetic agents, tamarindus indica seed gum, TEA-alginate, TEA-dextrin octenylsuccinate, tetradecyleicosanoic acid, tetradecyloctadecanoic acid, tetradecyloctadecyl behenate, tetradecyloctadecyl myristate, tetradecyloctadecyl stearate, tetrasodium etidronate, Theobroma grandiflorum seed butter glyceryl esters, tocopheryl succinate methylglucamide, tremella fuciformis polysaccharide, triacontene/VP copolymer, 1-Tridecanol, tripropylene glycol, undeceth-40, undecylenoyl inulin, undecylenoyl xanthan gum, vinyl alcohol/vinylformamide copolymer, bis-vinyl dimethicone/dimethicone copolymer, bis-vinyldimethicone/peg-10 dimethicone crosspolymer, hydrogenated microcrystalline wax hydrotreated, welan gum, xanthan gum, zinc undecylenoyl hydrolyzed wheat protein.

The skincare compositions in accordance with the invention may also comprise microbial ingredients (e.g., filtrates, extracts and/or ferment from Saccharomyces, Lactobacillus) and/or microbial-based formulation(s) such as Lallemand's Lallhair® P&R, Lallbiome™ 300, Lallbiome™ 201, Lallskin® Hydra, and/or Lallskin® Zn.

The skincare compositions in accordance with the invention may also comprise crosslinked hyaluronic acid. This compound could be used, for instance, as a thickener to make a clear moisturizing hydrogel. Particular examples include, but art not limited to Hyacross™ Hyaluronic Acid Microbeads TG300 (sodium hyaluronate crosspolymer, Bloomage Biotechnology Corp. Ltd.) and Hylasome™ EG10 (sodium hyaluronate crosspolymer, Vantage Personal Care).

In embodiments the skincare composition according to the invention provides for at least one of the following skin-related benefits:
i. smoothing the appearance and/or reducing presence of fine lines and/or wrinkles;
ii. improving the appearance of bags under the eyes and facial wrinkles;
iii. protecting the skin (e.g., from the environment such as the sun, chemicals, etc.; protection from oxidative stress);
iv. whitening of age spots (e.g., providing lightening of brown aging spots for a more even tone of the skin);
v. mattifying the skin (i.e. making the skin less shiny);
vi. regenerating and/or rejuvenating the skin;
vii. repairing and/or healing the skin (e.g., wound healing);
viii. densifying the skin and/or improving one or more mechanical properties of the skin (firmness, tone, elasticity, and/or flexibility)
ix. improving suppleness of the skin;
x. creating a protective film on the skin, e.g. a long lasting protective film lasting at least 24 hours, or at least a week, or at least ten (10) days;
xi. creating on the skin a film almost dry to the touch (e.g., a perceivable but very light film feeling on the superficial surface of the skin);
xii. creating a cooling sensation on the skin during application;
xiii. hydrating and/or moisturizing the skin;
xiv. preventing water evaporation from the skin;
xv. improving skin barrier integrity;
xvi. reducing epidermal water loss and/or improving skin water retention; and
xvii. non-comedogenic.

In embodiments, the skincare composition according to the invention consists of an instant skin tightener. In embodiments, the skincare composition provides immediately noticeable skin appearance benefits. For instance, the skincare composition according to the invention preferably dramatically improve the appearance of bags under the eyes and facial wrinkles.

In embodiments, a skin tightener composition in accordance with the present invention comprises biopolymer molecules as defined herein in combination with any suitable skin tightener compound. For instance, a skin tightener composition in accordance with the present invention may comprise one or more of the followings: sodium silicate; polyol(s) such as glycerin, propylene glycol, and butylene glycol; clay(s) such as bentonite, magnesium aluminum silicate, lithium magnesium sodium silicate and sodium magnesium fluorosilicate; soft focus powder(s) such as spherical silica, cellulose, polymethylsilsesquioxane, dimethicone/vinyl dimethicone crosspolymer; dimethylacrylamide/acrylic acid/polystyrene ethyl methacrylate copolymer and polyurethane-2, polymethyl methacrylate; polysaccharide(s) such as xanthan gum; resin(s) such as sodium polystyrene sulfonate; natural-based polymer(s) such as pullulan, egg albumin, gliadin, acacia senegal gum, rhizobian gum, hydrolyzed wheat protein/pvp, hydrolyzed chenepodium quinoa seed, hydrolyzed sesame protein pg, and porphyridium polysaccharide-frutarom; synthetic polymer(s) such as sodium polystyrene sulfonate, polyacrylate 21, adipic acid/neopentyl glycol crosspolymer, hdi/trimethylol hexyllactone cross polymer; humectant(s).

A skin tightener composition in accordance with the present invention may also comprise biopolymer molecules as defined herein and be mixed with, and/or used in combination with a two-part formulation that is activated when the two parts are combined. This includes for instance products comprising a prepolymer and an activating catalyst that promotes polymer cross-linking or polymerization. One particular example includes Neotensil™. Accordingly, the present invention encompasses skincare kits comprising two-part formulations that are activated when combined, wherein at least one of the formulation comprises biopolymer molecules as defined herein.

The skincare composition according to the invention may also find uses in additional skin-related applications, including but not limited to wound healing, regulation of sebum excess, prevention and/or treatment of acne, antimicrobial activity, providing beneficial antioxidants and/or antiradicals to the skin, as well as many other useful film-forming protection activities.

It is within the skills of those in the art to assess any formulate skincare compositions in accordance with the present inventio that balance performance and irritation potential with good product skin appearance, feel, and comfort. The skilled person can also formulate skincare compositions that will satisfy desirable key performance parameters such as film durability over time, cracking, flaking, whitening, peeling, minimizing skin shine, ease of use, easy uniform product application, etc.

It is also within the skills of those in the art to assess any potentially useful use of the skincare compositions according to the present invention. This may involve for instance carry out trials with multiple patients and/or using specialized equipment or tests to measure the skin conditions (e.g., hydration, color, smoothness, thickness, pH, transepidermal water loss measurement (TEWL), comedogenic and irritancy, etc.

### Methods of Manufacture and of Substitution of Microplastics

Another aspect of the present invention concerns methods of manufacturing skincare compositions as defined herein. A related aspect concerns the use of a biopolymer as described herein for replacing microplastics in skincare compositions.

In one embodiment, the method comprises substituting at least a portion of microplastics that may be present in skincare compositions with a biopolymer, e.g. a biopolymer suspension, as defined herein. In embodiments the biopolymer suspension comprises a suspension of insoluble and/or semi-soluble particles stably dispersed within a polar solvent.

In one embodiment, the method of manufacturing the skincare composition, and/or method for replacing microplastics in a skincare composition, comprises: (1) providing a biopolymer suspension comprising a suspension of insoluble and/or semi-soluble particles stably dispersed within a polar solvent; (2) mixing the biopolymer suspension with at least one of an humectant, an emollient, an occlusive, a ceramide, an emulsifier, a thickening agent, a preservative, an antioxidant, a fragrance, a functional ingredient, a UVB filter, a UVA filter, and a broad-spectrum filter. Of course skincare composition according to the invention is formulated (e.g., selection of ingredients, ratio, etc.) to obtain skincare composition which is dermatologically safe for repeated applications to human skin.

Those skilled in the art will recognize, or be able to ascertain, using no more than routine experimentation, numerous equivalents to the specific procedures, embodiments, claims, and examples described herein. Such equivalents are considered to be within the scope of this invention and covered by the claims appended hereto. The invention is further illustrated by the following examples, which should not be construed as further or specifically limiting.

### EXAMPLES

### Example 1: Manufacturing of Seven (7) Different Skincare Formulations

Seven different biopolymer formulations (referred herein as samples #1 to #7) were manufactured for the purpose of assessing their skincare properties. In samples #1 to #4 and #6 the biopolymer was chitosan. In samples #5 and #7, the biopolymer was cellulose.

### Sample #3 CMCB3.1

Fungal chitosan was processed using a two-step process as follow.

Step 1: The chitosan was milled for 150 mins in isopropanol using a CMC Basket Mill BM-100™ to reduce particle size. The general milling conditions were 1600 FPM (feet per minute) milling shaft speed and 15 Hz mixing shaft speed 75% media load of 1.4-1.7 mm zirconia beads, where 1 kg of powder was processed in isopropanol with a 20% w/w solid content to obtain a slurry. The slurry was next dried to remove any isopropanol to obtain a refined powder. That refined powder was used in the next step.

Step 2: The dried chitosan (785 g) was milled with 13 kg of distillated water for 150 mins using a 1.5 L Supermill Plus™ (a flow through horizontal mill), to yield a suspension referred herein as the “CMCB3.1 Chitosan suspension”. The general milling conditions to obtain that suspension were 2400 FPM (feet per minute) rotation speed with a pump flow rate of 5.5 GPH (gallons per hour) using 1.1 L of 1.25 mm zirconia beads, where 14 kilos of slurry was processed with a 7.18% w/w solids content.

### Sample #1 2022111602

A fatty alcohol chitosan suspension was generated by milling together the CMCB3.1 Chitosan suspension (30.1 g), glyceryl stearate (0.104 g) and cetyl alcohol (0.102 g) at 670 RPM with fifty units of 10 mm ball using the 10+1 Alt method, where it is milled for ten minutes followed by a pause for one minute then milling for ten minutes in the opposite direction for a total of 1 hour.

### Sample #4 2022111603

A fatty alcohol beeswax chitosan suspension was generated by milling together the CMCB3.1 Chitosan suspension (30.1 g), glyceryl stearate (0.100 g), cetyl alcohol (0.103 g) and beeswax (0.1 g) at 670 RPM with fifty units of 10 mm ball using the 10+1 Alt method, where it is milled for ten minutes followed by a pause for one minute then milling for ten minutes in the opposite direction for a total of 1 hour.

### Sample #2 2022111604

A fatty alcohol beeswax chitosan suspension was generated by milling the CMCB3.1 Chitosan suspension (30.0 g), glyceryl stearate (0.109 g), cetyl alcohol (0.103 g) and beeswax (0.2 g) at 670 RPM with fifty units of 10 mm ball using the 10+1 Alt method, where it is milled for ten minutes followed by a pause for one minute then milling for ten minutes in the opposite direction for a total of 1 hour.

### Sample #6 2022112103

A fatty alcohol beeswax chitosan suspension was generated by milling together the CMCB3.1 Chitosan suspension (30.0 g), glyceryl stearate (0.154 g), cetyl alcohol (0.145 g) at 670 RPM with fifty units of 10 mm ball using the 10+1 Alt method, where it is milled for ten minutes followed by a pause for one minute then milling for ten minutes in the opposite direction for a total of 1 hour. This was followed by the addition of beeswax (0.345 g) and further milling at 670 RPM with fifty units of 10 mm ball using the 10+1 Alt method.

### Sample #5 CMC B1.C

Alpha cellulose (Sigma Aldrich) was milled in distilled water using a 1.5 L Supermill Plus™. The general milling conditions were 2400 FPM rotation speed with a pump flow rate of 7.3 GPH using 982 mL of 1.4-1.7 mm zirconia beads, where 10 kilos of suspension were processed in a 5% w/w solids content (0.5 kg of cellulose and 9.5 kg water). Cellulose was suspended through the milling process while milling for 140 mins, producing a suspension.

### Sample #7 CMC B2C

Cellulose was processed using a two-step process.

Step 1: Alpha cellulose (Sigma Aldrich) was milled for 23 hours in isopropanol using a 1.5 L Supermill Plus™ to reduce particle size. The general milling conditions were 2750 FPM rotation speed with a pump flow rate of 10 GPH using 982 ml of 1.25 mm zirconia beads, where 10 kilos of slurry were processed at a 10% w/w solids content (1 kg of cellulose and 9 kg isopropanol). The slurry was next dried to remove any isopropanol and to obtain a refined powder which was then used in the next step.

Step 2:1 kg of dry cellulose from step 1 was milled in 9 kg of distilled water for 435 mins using the 1.5 L Supermill Plus™ to yield a suspension. The general milling conditions were 2400 FPM rotation speed with a pump flow rate of 11 GPH using 956 mL of 1.25 mm zirconia beads, where 10 kilos of slurry were processed at a 10% w/w solids content.

### Example 2: Testing of Cosmetic Properties of the Seven Different Formulations of Example 1

### SUMMARY

A home self-assess evaluation test was performed to determine the cosmetic properties of seven (7) different cream formulations manufactured at Example 1. The evaluation was based on visual and sensory self-assessment of a human female subject. Subject did not put any hydrating product on her skin 72 hours prior to the test and did not use any other product during the test. Hands were washed, as an any normal day, at least 2 times per day with mild soap. Hands were also being exposed to water and soap during daily shower.

### Test Details

The objective of the test was to assess hydration, wrinkles disappearance (film forming effect) and sensory feeling during applications of the cream samples on the skin. The test was carried out for nine (9) days using the following experimental parameters:

| Number of | | | Ambient | Time of | # Application | Site of | Type of | Qty applied | Type of |
|---|---|---|---|---|---|---|---|---|---|
| participants | Age | Sex | temp. | application | in 24 hrs | test | skin | per use | application |
| 1 | 55 | F | 19.5° C. | morning | 2 | back of | Dry | 0.3 g | superficial |
| | | | | & night | | the hand | | | |

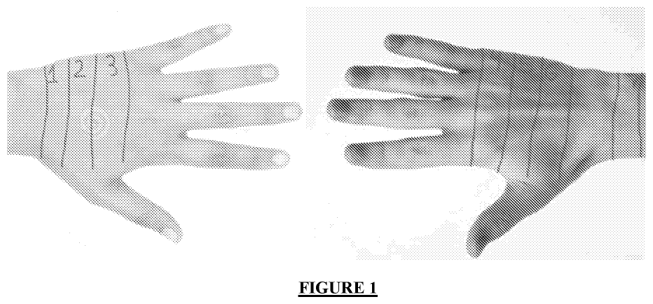

A predetermined amount of each cream sample was applied onto the back of the hand with a stainless spatula by following the scheme of application illustrated in FIG. 1. Once the cream was transferred onto the skin with the spatula, the cream was spread with a finger and by making a back-and-forth movement onto the site, from top to bottom, 10 times.

Sensory properties were also evaluated by rubbing small amount of cream between the thumb and the forefingers to assess each sample for absorption rate, pilling and texture.

### Visual & Sensory Observation of the Samples Tested

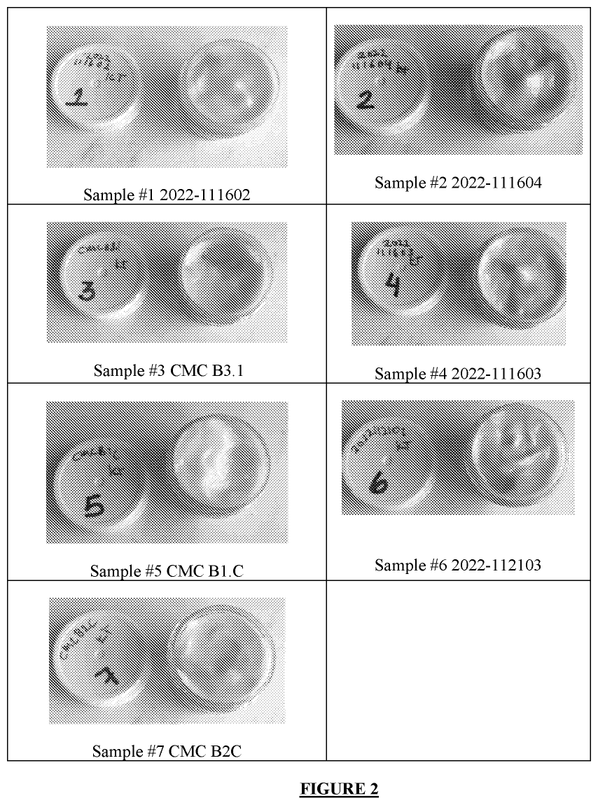

Visual and sensory observations of the seven tested samples are summarized in Table 1 below. Pictures of the samples at time TO are shown in FIG. 2.

TABLE 1
Visual and sensory observations of the seven samples tested

| | | Visual | | | | | Immediate |
|---|---|---|---|---|---|---|---|
| | | observation | | | | | observation |
| | | of sample | | | | | after |
| Sample | Application | in the jar | Color | Texture | Separation | Finger rubbing test | application |
| Sample #1: | Smooth | nice shiny | light | smooth, | jar is full, but | very light slippery feeling, | Feeling of |
| 2022-111602 | application, | texture | beige | very light | seems to | oil/silicone feeling, skin | wetness after |
| | very fast | | | watery, light | have some | seems to absorb a large | 30 minutes on |
| | absorption no | | | medication | separation | quantity, sandy residue on | the finger |
| | pilling | | | odor | around the | skin that seems to melt in | |
| | | | | | jar lid | the skin after a while, very | |
| | | | | | | light hydrating sensation. | |
| Sample #2: | application is | shiny light | light / | between a | light | smoother sensation, slower | Good |
| 2022-111604 | difficult, slow | texture | medium | gel and a | separation | absorption feeling; more | hydrating |
| | absorption | | beige | cream, | occurring at | like a cream, skin seems to | feeling after 30 |
| | and lots of | | | seems more | the top | absorb a far quantity, no | minutes. |
| | pilling | | | consistent | | residue on skin, skin seems | |
| | immediately | | | medium + | | to be filled with water under | |
| | but still able | | | medication | | a coating, cold sensation | |
| | to force into | | | odor | | after application, fingers | |
| | the skin. | | | | | even seem to be visually | |
| | | | | | | bulkier after application. | |
| | | | | | | More hydrated sensation | |
| | | | | | | after application | |
| Sample #3: | Smooth | observe | medium | very watery, | high | fare absorption rate, heavy | Heavy piling |
| CMC B3.1: | application, | separation | beige | light odor | separation in | pilling at the end, seems to | on skin after |
| | light white | after a couple | | | the jar | stay at the top of the skin | application. |
| | deposit on | of days, | | | | and not absorb but dry | |
| | skin, fast | seems to be | | | | during the application, | |
| | absorption | more each | | | | heavy pilling at the end, | |
| | | day | | | | smooth finish on skin. | |
| Sample #4: | Smooth | shiny, texture | light | nice | very light | never seems to penetrate, | Good |
| 2022-111603 | application, | seems a little | beige | consistency, | separation, | cold sensation during | hydrating |
| | fast | creamier | | with very | observation | application, a little sandy | feeling after 30 |
| | absorption no | | | light | of a little crust | feeling follows by a | minutes of |
| | pilling, dries | | | unevenness | on the side of | smoother feeling to finish | application |
| | easily | | | in the jar | the jar and in | with a dry sensation and | |
| | | | | | the lid, | heavy pilling | |
| | | | | | seems to dry | | |
| | | | | | rapidly | | |
| Sample #5: | Smooth | very shiny | almost | shiny | no visual | Three (3) stage application, | A little cold, |
| CMC B1.C | application, | gel-like | white | texture, light | separation, | feels watery and non- | absorbs very |
| | very fast | texture | | cream, most | very subtle | slippery at first, then you | fast at first, |
| | absorption a | | | interesting | smell | feel a smooth slippery effect | some piling at |
| | little pilling | | | texture | | and finish with dry touch | the end. |
| | | | | visually | | feeling. | Cooling effect. |
| | | | | | | | Application |
| | | | | | | | feeling is |
| | | | | | | | good. |
| Sample #6: | Smooth | Shiny watery | light | creamy | a medium | seems to absorb slowly, | leaves an |
| 2022-112103 | application, a | texture | beige | visually | separation at | | unpleasant dry |
| | little pilling, | | | | the top of the | | non-slippery |
| | leaves skin | | | | jar | | skin surface |
| | soft and | | | | | | after |
| | hydrated. | | | | | | application, |
| | Creamier at | | | | | | noticeable |
| | application | | | | | | cold |
| | | | | | | | sensation, |
| | | | | | | | very little |
| | | | | | | | pilling |
| Sample #7: | Smoother | shiny almost | light | dry very fast | some light | Feels watery at first but | the skin |
| CMC B2C | application, | creamy | grey / | on skin, | separation on | changes to a more | seems denser |
| | very fast | texture, much | white | different | top of the jar. | hydrating smooth | and almost |
| | absorption | lighter color | | smells, less | | sensation, good application | plumpier. |
| | very light | | | medication | | sensation. Pilling at the end | |
| | pilling | | | | | when start drying | |

### Quantitative Assessment of the Cream Samples

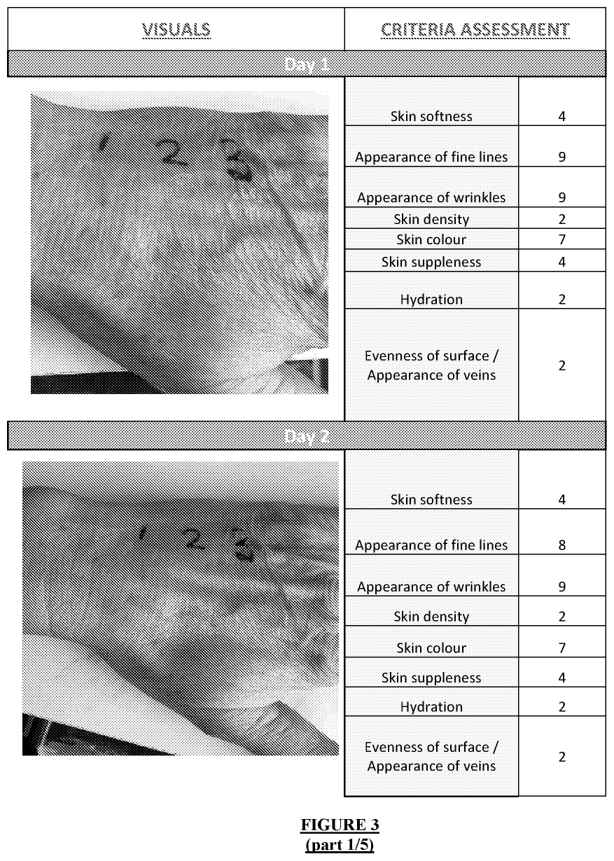

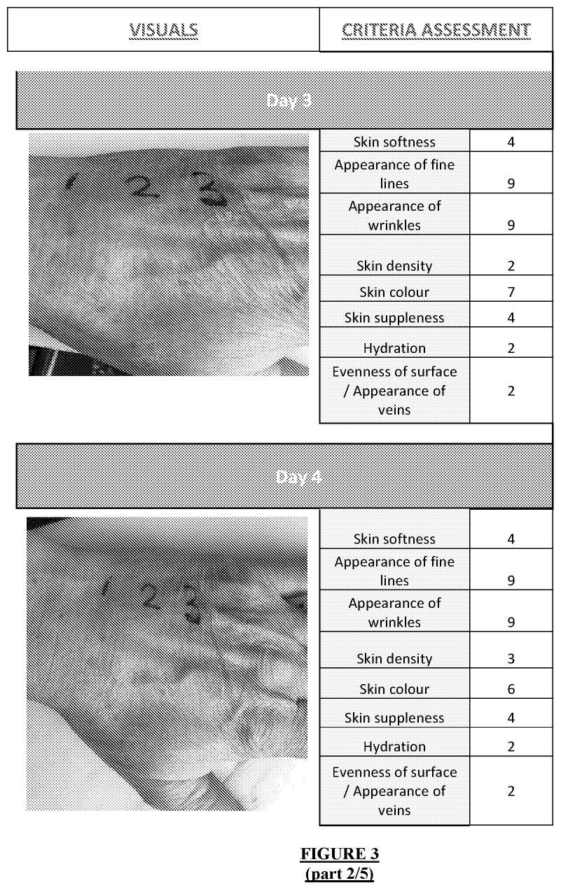

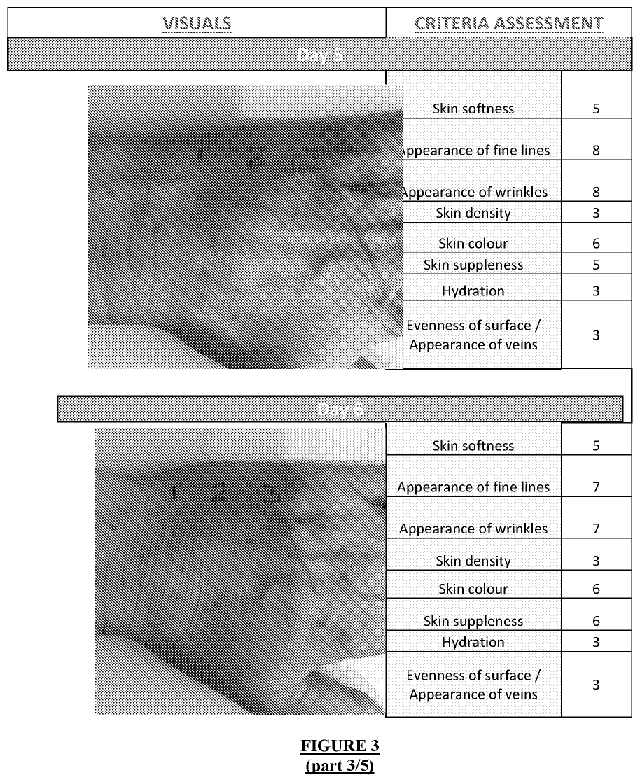

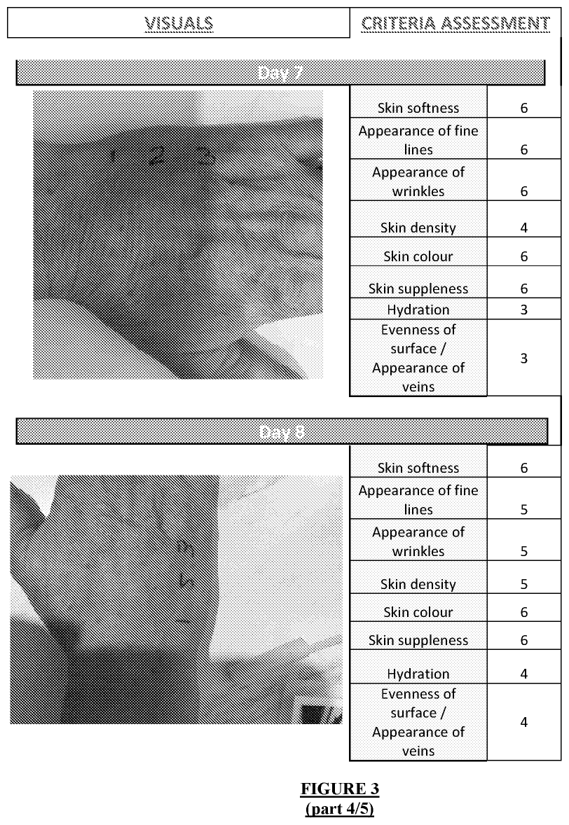

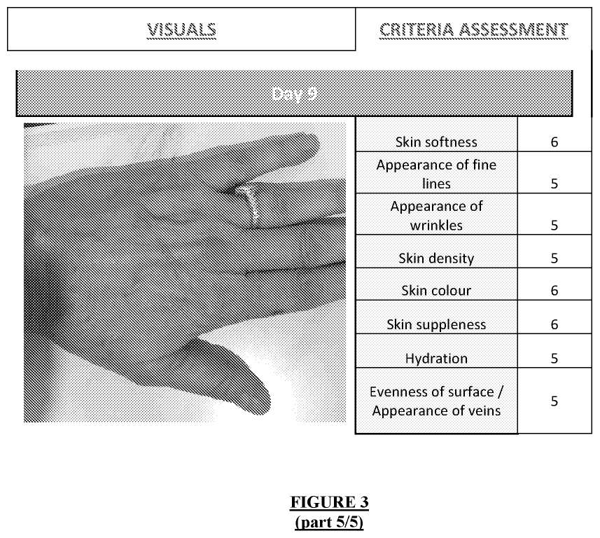

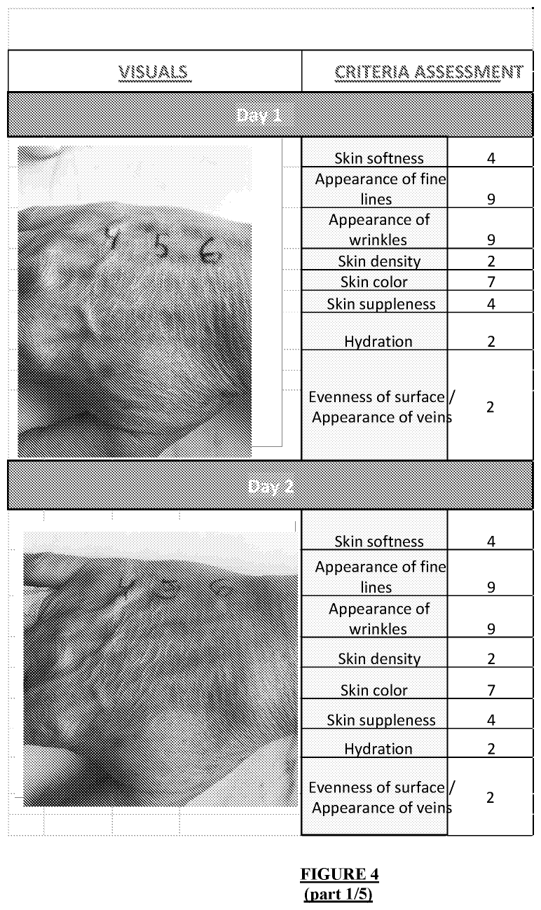

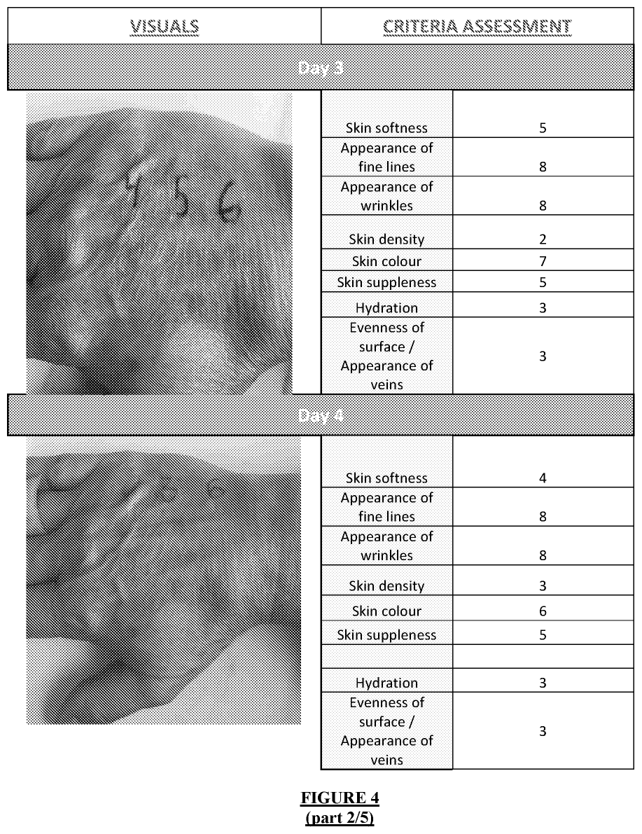

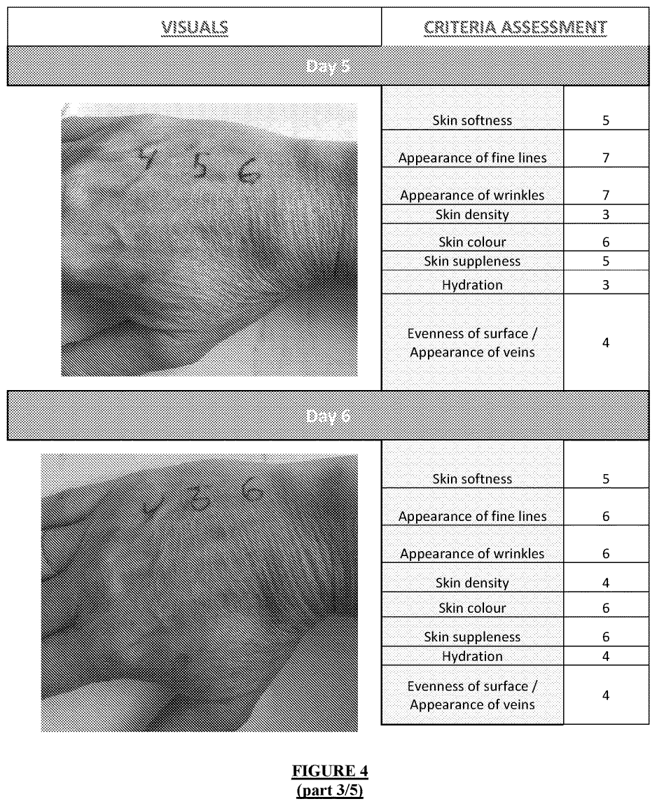

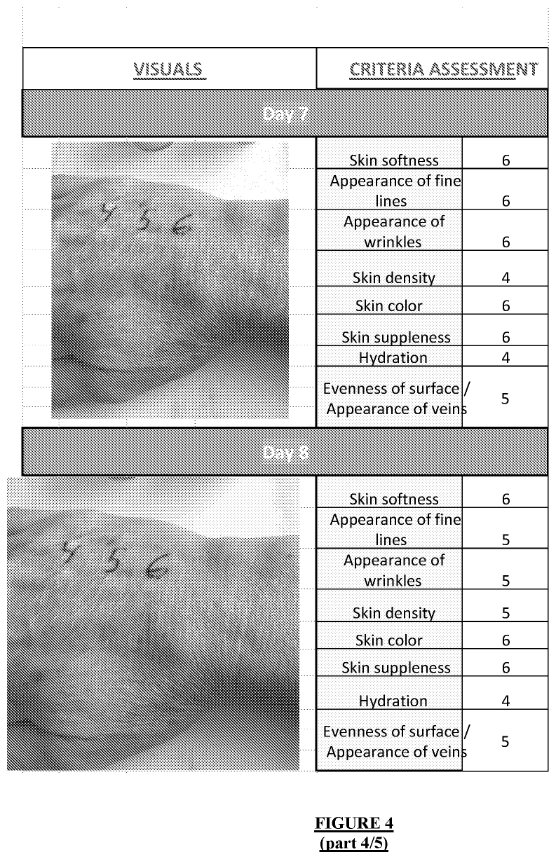

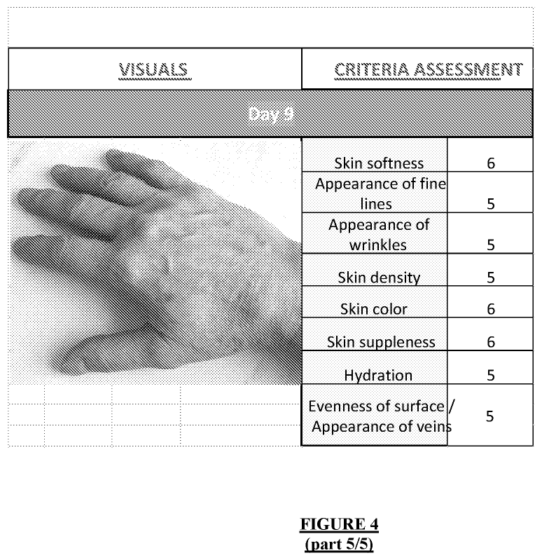

The samples were assessed during the nine days of the test and each day, they were given a score from 1 to 10 on many visual and sensory aspects. The measured observations for samples 1 to 3 are illustrated and summarized in FIG. 3. The measured observations for samples 4 to 6 are illustrated and summarized in FIG. 4.

### Results and Discussion

The purpose of the test was to verify if any and/or all the cream samples had film forming properties and whether these samples can smooth the appearance of the fine lines and wrinkles on the back of the hand. The purpose of the test was also to evaluate how much product was necessary to obtain visual results and for how long the results could last.

Based on self-assessment of the subject, all samples have demonstrated film forming properties. All samples have also demonstrated the ability to smooth the skin surface, leaving the skin very soft to the touch following application of the creams.

A very unusual and interesting effect was observed with the tested creams, in comparison to traditional skincare products readily available on the market. When applied onto the skin most skincare products leave a moist/sticky, shiny, feel and touch. In contradistinction, the samples tested in the present test left a perceivable and very light film feeling on the superficial surface of the skin. This film was almost dry to the touch. The surface of the skin did not feel moist and hydrated, and it was completely mat. However, the skin underneath this tiny “protective” film was visually softer, denser, almost plumped and much more supple. Fine lines and wrinkles were less visible and even seemed to have been repaired.

Sample #2 (2022-111604) seems to be an excellent candidate for smoothing the appearance of not only fine lines but also wrinkles. Samples #1 (2022-11602) and #4 (2022-111603) were also great candidates.

Sample #4 (2022-111603) and sample #5 (CMC-B1C) were creamier during application but they produced some piling with an extensive friction during application. The absorption rate was also very fast. Pilling was also observed with sample #3 (CMC-B3.1) when applied to a larger area.

Sample #7 (CMC-B2C) demonstrated some very special properties: during application and a few minutes thereafter, a cooling sensation was easily observed. There was also a be rapid augmentation of the density of the skin. Such properties could potentially be extremely interesting in the fight against age damages, and to address density loss in areas like the neck, the hands, and the eye contour.

A subtle, not unpleasant, smell of medication was observed with most samples. That smell disappeared rapidly during application and no residual odour was observed after application. This subtle smell could be a good way to differentiate the skincare products of the invention from commercially available products, since the latter typically contain strong perfume odors that are often not appreciated by customers.

Most samples, except #5 (CMC-B1C), showed different grades of phase separation, samples #4 (2022-111603) and #7 (CMC-B2C) displaying a lesser grade.

It was also observed that once the films had dried onto the skin, they were somewhat “persistent” since they were not washed away with simple rinsing or mild soap. The protective films lasted over 24 hours. Presence of such film likely contributes to keep a natural hydration of the skin, e.g., by preventing water evaporation from the skin.

Also, a whitening effect was observed with at least 3 of the samples (#2, #4 and #5). After application of these samples, the skin had an even tone and existing brown aging spots seemed to be much lighter (see FIGS. 3 and 4).

### Example 3: Manufacturing of Two (2) Additional Skincare Formulations

Four (4) additional biopolymer formulations (referred herein as samples #8 #9, #10 and #11) were manufactured for the purpose of assessing their skincare properties.

### Sample #8—Cellulose/Chitosan/Beeswax-Based-Based Composition [Batch DEF23051502]

The cellulose/chitosan/beeswax-based-based composition comprised 5.7% w/w mushroom chitosan, 0.43% w/w alpha cellulose, 5% w/w Dermosoft™ pentiol ecoby (Evonik), 0.55% cetyl alcohol, and 0.93% beeswax [Batch DEF23051502].

The composition was prepared as follow. First, an original batch “milled formula” comprising alpha cellulose, chitosan and water was milled in a NETZSCH LabStar Zeta Small Media Mill™ for 110 minutes (PSI of 6.9-11, agitator speed 3000 rpm, chiller turned on to maintain temperature of 32° C.). Separately, the following “oily mixture” mixture was made by melting together at 57° C. cetyl alcohol, beeswax synthetic pellets and Dermosoft™. The oily mixture was mixed until the temperature reduced to 52° C. The milled formula (total of 385 grams) was added ⅙th at a time to the oily mixture. Both were mixed together until the temperature went down to 45° C. Once homogenously mixed, this resulting mixture was added very slowly into the ball miller with the remaining of the original batch of milled formula at the 110 min mark. Milling was then continued for 10 further minutes.

### Sample #9—ChitoCellDF™ [DEF23041801]

A cellulose/chitosan-based composition in accordance with the invention comprising 5% w/w mushroom chitosan, 0.4% w/w cellulose, 5% w/w Dermosoft™ and water was prepared [Batch DEF23041801]. Briefly the chitosan and cellulose were wet milled with water for 30 minutes (agitator pressure 7-9 PSI; agitator speed 3000 rpm). The Dermosoft™ preservative was added at 30 minutes, and the milling was continued for another 60 minutes. The temperature of the mixture was maintained between about 30° C.-40° C.

### Sample #10—ChitoCellDF™ [DEF23110702]

A cellulose/chitosan-based composition in accordance with the invention was prepared like for the other samples above. The composition comprised water, 5% w/w mushroom, 5% w/w Dermasoft™, 3% w/w Vegetal glycerin, 2.5% w/w beeswax, 2% w/w cetyl alcohol, 2% w/w adifyline, 1.5% w/w sepilift, 0.65% w/w RoseHip Oil, 0.4% w/w cellulose (INCI: water, whitosan, pentylene glycol, beeswax, glycerin, cetyl alcohol, acetyl hexapeptide 38, dipalmitoyl hydroxyproline, Rosa moschata seed sil, cellulose).

### Sample #11—ChitoCellDF™ [DEF23110901]

A cellulose/chitosan-based composition in accordance with the invention was prepared like for the other samples above. The composition comprised water, chitosan 5% w/w, Dermosoft 5% w/w, oilvem 1000 4% w/w, argireline 4% w/w, jojoba butter 3% w/w, beeswax 2.5% w/w, cetyl alcohol 1% w/w (INCI: chitosan, beeswax, cetyl alcohol, pentylene glycol, Simmondsia chinensis oil, hydrogenated vegetable oil, tocophero, acetyl hexapeptide-8, sodium benzoate, cetearyl olivate, sorbitan olivate).

### Example 4: Evaluation of Wrinkles and Fine Lines Efficacy

### SUMMARY

The cellulose/chitosan-based composition identified as Sample #9 in Example 3 (i.e., Batch DEF23041801], was tested in vivo in order to evaluate the wrinkles and fine lines efficacy on 11 healthy male and female volunteers aged between 18 and 65 years old. The composition was evaluated using standard guidelines by an independent third-party laboratory.

### Test Details

This study is aimed to evaluate the efficacy of the product in improving wrinkles and fine lines using the VisioTOP-300™ with AEVA-V4™ software system (EOTECH, France) in normal usage conditions after 15 minutes of the first application and using the product twice a day, daily, for a period of 10 Days±2 days. 3D images were used to monitor the effects of the product with the VisioTOP-300™ before product usage (TO), after 15 minutes of the first application (T15 Min) as well as after 10 days of product usage (T10 Days±2 days).

The product was used after the first measurement (TO), on the volar surface of the face, twice a day, daily, for a period of 10 days (T10 Days±2 days).

The measurements were done by the instrument at each experimental time (TO, T15 Min and T10 Days±2 days) at the level of the treated area. The skin point analyzed at different times were made on a randomized side of the face for each time of analysis. The area was evaluated in each macrophotograph using the VisioTOP-300™

The distribution of the values obtained during the measurements at the various experimental times for the treated area with the product were compared with an analysis (T15 Min versus TO and T10 Days±2 days versus TO) using Student's t test. Values of p<0.05 were considered significant.

Crow's Feet evaluation was used for assessing the effectiveness of the product. The most representative parameters for the anti-wrinkle efficacy evaluation, i.e., Ra and Rz were also measured by the instrument, where Ra is the linear average profile roughness and Rz is the average maximum profile height difference.

### RESULTS AND CONCLUSIONS

The product was highly effective in improving crow's feet roughness and wrinkles. Indeed, the study observed a statistically significant reduction of winkles upon application of the tested composition.

Briefly, the mean Rz parameter (Average Maximum Profile Height Difference) demonstrated a significant decrease by an average value equal to −5.3% after 15 minutes of product application (T15 Min) and demonstrated a significant decrease by an average value equal to −7.5% after 10 days of product usage (T10 Days±2 days), respecting the mean value measured at TO (before application of the product). These results are shown in Table 2 and Table 3.

TABLE 2
Mean Rz on Crow's Feet Zone

| Time | Mean values |
|---|---|
| T0 | 0.0381 |
| T15 Min | 0.0361 |
| T10 Days ± 2 days | 0.0353 |

TABLE 3
Mean % variation of Rz and p value of Student's test

| TIME | Mean % variation | p-value |
|---|---|---|
| T15 Min vs T0 | −5.3% | 0.0039 |
| T10 Days ± 2 days vs T0 | −7.5% | 0.0020 |

In particular, the mean Ra parameter (Linear Average Profile Roughness) demonstrated a significant decrease by an average value equal to −2.6% after 15 minutes of product application (T15 Min) and demonstrated a significant decrease by an average value equal to −3.1% after 10 days of product usage (T10 Days±2 days), respecting the mean value measured at TO (before application of the product). The means of Ra average on Crow's Feet zone are shown in Table 4.

TABLE 4
Mean Ra on Crow's Feet Zone

| Time | Mean values |
|---|---|
| T0 | 0.0122 |
| T15 Min | 0.0119 |
| T10 Days ± 2 days | 0.0118 |

Considering that P Values refer to a statistically significant variation of the Ra value when below 0.05 (p Value <0.05), the variation can be considered statistically significant, according to the p Value analysis when comparing T15 Min to T0 (0.0291). The variation can be considered statistically significant, according to the p Value analysis when comparing T10 Days±2 days to T0 (0.0179). The mean % variation of Ra average and p value of Student's t test is shown in Table 5.

TABLE 5
Mean % variation of Ra and p value of Student's test

| TIME | Mean % variation | p-value |
|---|---|---|
| T15 Min vs T0 | −2.6% | 0.0291 |
| T10 Days ± 2 days vs T0 | −3.1% | 0.0179 |

Accordingly, these results clearly demonstrate that biopolymer compositions in accordance with the present invention are active and useful in reducing both wrinkles and fine lines.

### Example 5: Skin Barrier Effect and Water Resistance Evaluation-Cellulose/Chitosan-Based Composition

The objective of this study was to demonstrate the skin barrier effect and the waterproof effect of a cellulose/chitosan-based composition in accordance with the invention. The cellulose/chitosan-based composition is identified as Sample #9 in Example 3 (i.e., Batch DEF23041801]. The study involved 13 volunteers and the evaluation was done by two specialized technicians.

### Skin Barrier Effect

In the adopted experimental conditions, the product under examination demonstrated a significant decrease in the Transepidermal water loss (TEWL) values on the skin where the product was applied. The product showed a variation of −10.1% after T8 Hours±2 hours and of −16.4% after T12 Hours±2 hours (see Table 6). Boteh values were statistically significant when compared to TO.

TABLE 6
Variation of TEWL values

% Variation of mean TEWL values

| TIME | Batch DEF23041801 | Control |
|---|---|---|
| T8 Hours ± 2 hours | −10.1% | 1% |
| T12 Hours ± 2 hours | −16.4% | 0.8% |

Accordingly, these results clearly demonstrate that biopolymer compositions in accordance with the present invention have positive effects on skin barrier integrity and are effective for reducing epidermal water loss and/or improving skin water retention.

### Waterproof Effect

To assess the waterproof effect, a baseline pigment called Timica® Terra Brown was used for comparison with the cellulose-based composition under examination. The evaluation process included splashing water on the forearm area of the volunteers after applying the tested product (i.e., cellulose/chitosan-based composition as defined above). During the evaluation, two technicians assigned scores ranging from 1 to 10 based on the persistence of the tested product after splashing water 10 times. If the mean of these scores is equal to or greater than 7, the product is considered waterproof after splashing water 10 times from the initial application.

The two specialized technicians gave the tested product a mean score of 9.1 out of 10 for persistence. This high score indicates a high level of waterproof effect. Accordingly, the biopolymer composition in accordance with the present invention displays strong water proof effect since it can withstand ten water splashing after application on the skin.

Overall, these above results demonstrate that biopolymer compositions in accordance with the present invention have important skin barrier effects.

### Example 6: Skin Tolerance (Patch Test)

A test was carried out by an independent third-party laboratory to assess local skin tolerance of a biopolymer composition in accordance with the present invention. The composition was of the same batch than the cellulose/chitosan/beeswax-based composition identified as Sample #8 in Example 3 (i.e., Batch DEF23051502).

Briefly, about 0.07-0.1 ml of test product (i.e., biopolymer composition) was applied on the intact skin of the back of 14 adult volunteers. The product was kept in contact with the skin for 48 hours under semi-occlusive patch. Along with the test product a “negative” control empty patch was also applied. Effects caused by the application of the test product were observed 15 minutes and 24 hours after removal of the patch to assess skin reactions, considering parameters such as erythema, edema, dryness/desquamation, and vesicles, with severity scores ranging from 0 to 3.

The Mean Irritation Index (MII) of the test product was 0 at both time periods (15 min and 24 hours after patch removal). As such, the test product was classified as “not-irritant” and “dermatologically tested for skin compatibility”, according to the reference standard (data not shown). These results thus confirm that skincare biopolymer compositions according to the present invention are dermatologically safe.

Likewise, Sample #10 and Sample #11 (see Example 3 above) were submitted to an independent third-party laboratory to assess local skin tolerance and both were considered “non-primary irritant” after 48 hours in contact with the skin of volunteers.

### Example 7: Water Retention Effect-Cellulose/Chitosan/Beeswax-Based Composition

Trans Epidermal Water Loss (TEWL) is a very sensitive indicator of the skin barrier integrity and it can be measured instrumentally by means of evaporimetry.

This study was aimed to evaluate the effect of the cellulose/chitosan/beeswax-based composition identified as Sample #8 in Example 3 (i.e., Batch DEF23051502) on the TEWL after 8 hours and 12 hours of application on the surface of the skin. The evaluation implied the comparison of the analyzed area at T8 Hours±2 hours and T12 Hours±2 hours with the TEWL measured at TO (before the application of the product). Measurements were performed with a Tewameter® TM300 (Courage-Khazaka GmbH, Germany.

The evaluation involved thirteen (13) volunteers. The product was applied epicutaneously in a non-occlusive manner, massaging until absorbed. The amount of product applied was approximately 2 mg/cm², within 35 cm² area according to the Colipa Guidelines. During the 12 hours of the study, the volunteers were asked to not apply any other product and to not wash the area under examination. Before each measurement with the Tewameter®, each volunteer was allowed to relax for approximately 15 minutes in an air-conditioned room to avoid anomalous sampling due to excessive sweating or stress.

The testing of the product lasted 12 hours for each one of the 13 selected volunteers included in the study. The measurements with the Tewameter® were taken on the same treated area before the application of the product (T0), 8 hours after product application (T8 Hours±2 hours) and 12 hours after product application (T12 Hours±2 hours). The TEWL results are expressed as a mean percentage difference of the measured parameter at T8 Hours±2 hours and T12 Hours±2 hours in comparison with the measured parameter at T0. A statistical evaluation using Student's t test was performed in order to evaluate the significance of the observed differences (values of p<0.05 were considered significant).

In the adopted experimental conditions, the product under examination demonstrated a significant decrease the transepidermal water loss (TEWL) values on the skin where the product was applied. As displayed in Table 6 and Table 7 below, the product showed a variation of −12.7% after T8 Hours±2 hours and −19.0% after T12 Hours±2 hours with respect to T0. These variations were considered statistically significant (p values <0.05) when comparing T8 Hours±2 hours to T0 and when comparing T12 Hours±2 hours to T0.

TABLE 6
Mean TEWL values expressed in g/h/m²

| TIME | Batch DEF23051502 | Control |
|---|---|---|
| T0 | 8.78 | 8.75 |
| T8 Hours ± 2 hours | 7.66 | 8.7 |
| T12 Hours ± 2 hours | 7.10 | 8.73 |

TABLE 7
Percent variation of the mean TEWL values

| | Batch DEF23051502 | Control |
|---|---|---|
| T8 Hours ± 2 hours | −12.7 | −0.5 |
| T12 Hours ± 2 hours | −19 | −0.2 |

Accordingly, these results clearly demonstrate that biopolymer compositions in accordance with the present invention have positive effects on skin barrier integrity and are effective for reducing epidermal water loss and/or improving skin water retention.

### Example 7: Water Proof Effect-Cellulose/Chitosan/Beeswax-Based Composition

The objective of this study was to demonstrate the waterproof effect of a cellulose/chitosan/beeswax-based composition in accordance with the invention. The tested product was the cellulose/chitosan/beeswax-based composition identified as Sample #8 in Example 3 (i.e., Batch DEF23051502). The study involved 13 volunteers and the testing procedure was the same as for Sample #9 in Example 5.

In this study, the two specialized technicians gave the tested product a mean score of 9.7 out of 10 for persistence of the product after splashing water 10 times from initial product application. This high score indicates a high level of waterproof effect. Accordingly, the biopolymer composition in accordance with the present invention displays strong water proof effect since it can withstand ten water splashing after application on the skin.

### Example 8: Evaluation of Wrinkles and Fine Lines Efficacy

The cellulose/chitosan/beeswax-based composition identified as Sample #8 in Example 3 (i.e., Batch DEF23051502) was further tested in vivo in order to evaluate its efficacy on removing wrinkles and fine lines. The study involved 13 volunteers and the testing procedure was the same as for Sample #9 in Example 4.

Thee study demonstrated that the tested product was highly effective in improving crow's feet roughness and wrinkles. Indeed, the study observed a statistically significant reduction of winkles upon application of the tested composition.

Particularly, the mean Rz parameter (Average Maximum Profile Height Difference) demonstrated a significant decrease by an average value equal to −5.9% after 15 minutes of product application (T15 Min). This value was considered statistically significant compared to T0 (p Value=0.0027).

Accordingly, these results clearly demonstrate that biopolymer compositions in accordance with the present invention are effective in reducing both wrinkles and fine lines, and that this positive effect can be seen quickly after application (e.g., within 15 min.).

### Example 9: Evaluation of Comedogenicity

The cellulose/chitosan-based composition identified as Sample #9 in Example 3 (i.e., Batch DEF23041801], was further tested in vivo to measure how likely the product is to clog skin pores.

The composition was evaluated using standard guidelines by an independent third-party laboratory. The results of the study confirmed that the composition is “non-comedogenic”, i.e., that the composition did not clog or block the pores on the volunteers' skin. This is another great benefit of the invention biopolymer compositions in accordance with the present invention. Because they do not clog pores of the skin the present compositions may help reduce the chances of acne and prevent development of pimples.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “a compound” includes one or more of such compounds and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses, and such.

It is understood that the examples and embodiments described herein are for illustrative purposes only and that various modifications or changes in light thereof will be suggested to persons skilled in the art and are to be included within the present invention and scope of the appended claims.

1. A skincare composition comprising:
(i) a stable homogeneous aqueous suspension of insoluble and/or semi-soluble biopolymer particles stably dispersed within a polar solvent; and
(ii) at least one of a humectant, an emollient, an occlusive, a ceramide, an emulsifier, a thickening agent, a preservative, an antioxidant, a fragrance, a functional ingredient, a UVB filter, a UVA filter, and a broad-spectrum filter;
wherein said skincare composition is formulated for a dermatologically safe application to human skin; and
wherein said composition is effective for treating skin wrinkles when applied to human skin.

2. The skincare composition of claim 1, wherein said composition provides for at least one skincare benefit selected from the group consisting of:
(i) smoothing the appearance and/or reducing presence of fine lines and/or wrinkles;
(ii) improving the appearance of bags under the eyes and facial wrinkles;
(iii) protecting the skin;
(iv) whitening of age spots;
(v) mattifying the skin;
(vi) regenerating and/or rejuvenating the skin;
(vii) repairing and/or healing the skin;
(viii) densifying the skin and/or improving one or more mechanical properties of the skin;
(ix) improving suppleness of the skin;
(x) creating a protective film on the skin lasting at least 24 hours;
(xi) creating on the skin a film almost dry to the touch;
(xii) creating a cooling sensation on the skin;
(xiii) hydrating and/or moisturizing the skin;
(xiv) preventing water evaporation from the skin;
(XV) improving skin barrier integrity; and
(xvi) reducing epidermal water loss and/or improving skin water retention.

3. (canceled)

4. The skincare composition of claim 1, wherein the biopolymer is selected from the group consisting of chitin, chitosan, cellulose, hemicellulose, lignin, amylose, actin, fibrin, collagen, silk, fibroin, keratin, wool, alginic acid and mixtures thereof.

5. The skincare composition according to claim 1, wherein the biopolymer is selected from the group consisting of gelatin, pectin, starch, amylopectin, agarose, alginic acid, alginate, hyaluronic acid, RNA, DNA, xanthan gum, guar gum, latex, polymannans, suberin, cutin, cutan, and mixtures thereof.

6. The skincare composition according to claim 1, wherein the aqueous solvent is selected from the group consisting of: water, isopropanol, cetyl alcohol, ethanol, propanol, methanol, glycerol, acetic acid, and mixtures thereof.

7. The skincare composition according to claim 6, wherein the humectant is selected from the group consisting of glycerin, urea, hyaluronic acid, solicylic acid, alpha hydroxy acid, propylene glycol, honey, sorbitol, butylene glycol, sodium PCA, allantoin, sodium lactate, dicyanamide, tremella extract, seaweed, aloe vera and mixtures thereof.

8. The skincare composition according to claim 1, wherein the emollient is selected from the group consisting of petrolatum, lanolin, mineral oil, dimethicone and mixtures thereof.

9. The skincare composition according to claim 1, wherein the occlusive is selected from the group consisting of mineral oil, petrolatum, lanolin, dimethicone, shea butter and mixtures thereof.

10. The skincare composition according to claim 1, wherein the ceramide is selected from the group consisting of ceramide EOS, ceramide NS, ceramide NG, ceramide NP, ceramide AP, ceramide EOP, phytosphingosine, sphingosine and mixtures thereof.

11. The skincare composition according to claim 1, wherein the emulsifier is selected from the group consisting of glyceryl stearate, sodium lauryl sulfate, sodium dioctyl sulfosuccinate, tragacanthins, lecithin, mustard seed, soy lecithin, sodium phosphates, diacetyl tartaric acid ester of monoglyceride, sodium stearoyl lactylate, and mixtures thereof.

12. The skincare composition according to claim 1, wherein the thickening agent is selected from the group consisting of magnesium aluminium, silicate, silica, bentonite, carbomer, cetyl palmitate, ammonium acryloyldimethyltaurate and mixtures thereof.

13. The skincare composition according to claim 1, wherein the preservative is selected from the group consisting of phenoxyethanol, benzyl alcohol, sodium benzoate, potassium sorbate, ethylhexylglycerin, DMDM hydantoin, quaternium-15, imidazolidinyl urea, diazolidinyl urea, polyoxymethylene urea, sodium hydroxymethylglycinate, 2-bromo-2-nitropropane-1,3-diol (bromopol), glyoxal, ethylparaben, butylparaben, methylparaben, propylparaben, isobutylparaben, isopropylparaben, benzylate, benzoic acid, benzyl ester, methylisothiazolinone (MIT; 2-methyl-4-isothiazoline-3-one), methylchloroisothiazolinone (CMIT; 5-chloro-2-methyl-4-isothiazolin-3-one), phenoxyethanol, 2-phenoxyethanol, Euxyl K™400 (mixture of phenoxyethanol and 1,2-dibromo-2,4-dicyanobutane), triclosan (TSC), triclocarban (tcc), benzyl alcohol, benzalkonium chloride, citric acid, dehydroacetic acid, essential oils, grapefruit seed extract, lactic acid, levulinic acid, potassium sorbate, sodium dehydroacetate, sodium metabisulfite, sodium salicylate, sorbic acid, vitamin E, zinc pyrithione and mixtures thereof.

14. The skincare composition according to claim 1, wherein the antioxidant is selected from the group consisting of vitamin A, vitamin C, vitamin E, vitamin B3, green tea extract, resveratrol, curcumin, lycopene, coenzyme Q10, polyphenols, C. asiatica extract, tamanu oil, and mixtures thereof.

15. The skincare composition according to claim 1, wherein the fragrance is selected from the group consisting of linalool, citronellol, limonene, geraniol, eugenol, lavender oil (Lavandula angustifolia), rose flower extract (Rosa damascena), bergamot oil (Citrus bergamia), ylang-ylang oil (Canaga odorata), lemon (Citrus limon), lime (Citrus aurantifolia or Citrus medica), orange (Citrus sinensis), tangerine (Citrus tangerine), peppermint (Mentha piperita), spearmint (Mentha spicata), eucalyptus, cinnamon (cinnamomum)/, cinnamal and mixtures thereof.

16. The skincare composition according to claim 1, wherein the functional ingredient is selected from the group consisting of biological extracts, vitamins, amino acids, peptides, proteins, mycosporines, Mycosporine-like Amino Acids (MAAs), imino compounds that absorb ultraviolet radiations, essential fatty acids, hydroxy acids, beta-glucans and mixtures thereof.

17. The skincare composition according to claim 1, with the proviso the stably dispersed biopolymer molecules have not been obtained by solubilisation into an acidic solution or by solubilisation into a basic solution.

18. The skincare composition according to claim 1, wherein said skincare composition is selected from the following compositions:
Composition A:

| | % (w/w) IN THE |
|---|---|
| FUNCTION(S) | FORMULATION |
| biopolymer | 0.3-10 |
| (dry weight) | |
| humectant | 0-20 |
| emollient | 1-15 |
| occlusive | 0-10 |
| ceramide | 0-5 |
| emulsifier | 0.2-10 |
| thickening | 0-5 |
| agent | |
| preservative | 0.5-5 |
| antioxidant | 0-0.5 |
| fragrance | 0-0.2 |
| functional | 0-10 |
| ingredient | |
| UVB filter | 0-20 |
| UVA filter | 0-20 |
| broad- | 0-20 |
| spectrum filter | |
| Water | 30-95 |
| (to complete to | |
| 100%) | |

Composition B:

| | % (w/w) IN THE |
|---|---|
| FUNCTION(S) | FORMULATION |
| biopolymer | 5-10 |
| (dry weight) | |
| preservative | 0.5-5 |
| Water to make | 85-94.5 |
| 100% | |

Composition C:

| | % (w/w) IN THE |
|---|---|
| FUNCTION(S) | FORMULATION |
| biopolymer | 5-10 |
| (dry weight) | |
| stabilizer | 0.2-5 |
| Water to make | 85-94.8 |
| 100% | |

Composition D:

| | % (w/w) IN THE |
|---|---|
| FUNCTION(S) | FORMULATION |
| biopolymer | 5-10 |
| (dry weight) | |
| emollient | 0.2-1 |
| preservative | 0.3-5 |
| stabilizer | 0.2-1 |
| Water to make | 83-94.3 |
| 100% | |

Composition 3:

| | % (w/w) IN THE |
|---|---|
| Ingredient | FORMULATION |
| chitosan | ~7.2 |
| (dry weight) | |
| water | ~92.4 |
| sodium | ~0.4 |
| benzoate and | |
| potassium sorbate | |

Composition 1:

| | % (w/w) IN THE |
|---|---|
| Ingredient | FORMULATION |
| chitosan | ~7.2 |
| (dry weight) | |
| water | ~91.8 |
| glyceryl | ~0.3 |
| stearate | |
| cetyl alcohol | ~0.3 |
| sodium | ~0.4 |
| benzoate and | |
| potassium sorbate | |

Composition 4:

| | % (w/w) IN THE |
|---|---|
| Ingredient | FORMULATION |
| chitosan | ~7.1 |
| (dry weight) | |
| water | ~91.5 |
| glyceryl | ~0.3 |
| stearate | |
| cetyl alcohol | ~0.3 |
| beeswax | ~0.3 |
| sodium | ~0.4 |
| benzoate and | |
| potassium sorbate | |

Composition 2:

| | % (w/w) IN THE |
|---|---|
| Ingredient | FORMULATION |
| chitosan | ~7.1 |
| (dry weight) | |
| water | ~91.8 |
| glyceryl | ~0.4 |
| stearate | |
| cetyl alcohol | ~0.3 |
| beeswax | ~0.7 |
| sodium | ~0.4 |
| benzoate and | |
| potassium sorbate | |

Composition 6:

| | % (w/w) IN THE |
|---|---|
| Ingredient | FORMULATION |
| chitosan | ~7.0 |
| (dry weight) | |
| water | ~90.5 |
| glyceryl | ~0.5 |
| stearate | |
| cetyl alcohol | ~0.5 |
| beeswax | ~1.1 |
| sodium | 0.4 |
| benzoate and | |
| potassium sorbate | |

Composition 5:

| | % (w/w) IN THE |
|---|---|
| Ingredient | FORMULATION |
| Cellulose | 5 |
| (dry weight) | |
| water | 95 |

Composition 7:

| | % (w/w) IN THE |
|---|---|
| Ingredient | FORMULATION |
| Cellulose | 10 |
| (dry weight) | |
| water | 90 |

Composition 8:

| | % (w/w) IN THE |
|---|---|
| Ingredient | FORMULATION |
| chitosan | ~5.7 |
| (dry weight) | |
| alpha cellulose | ~0.43 |
| preservative | ~5.0 |
| cetyl alcohol | ~0.55 |
| beeswax | ~0.93 |
| water to make | ~87.4 |
| 100% | |

Composition 9:

| | % (w/w) IN THE |
|---|---|
| Ingredient | FORMULATION |
| chitosan | ~5.0 |
| (dry weight) | |
| alpha cellulose | ~0.4 |
| preservative | ~5.0 |
| water to make | ~89.6 |
| 100%. | |

19. The skincare composition according to claim 1, wherein said skincare composition consists of an instant skin tightener.

20. The skincare composition according to claim 19, wherein said instant skin tightener further comprises one or more of the followings: sodium silicate; polyol(s); clay(s); soft focus powder(s); polysaccharide(s); resin(s); natural-based polymer(s); synthetic polymer(s) and humectant(s).

21. The skincare composition according to claim 1, wherein the skin is from the hands, breast, neck, arms, legs, eye contour, forehead, face, and body.

22. The skincare composition according to claim 1, wherein said skincare composition is formulated as a paste, an ointment, a cream, a lotion, a gel or a milk.

23. A method of skin protection, comprising applying onto the skin a skincare composition as defined in claim 1.

24. A method for treating skin wrinkles, comprising applying a composition as defined in claim 1, on a surface of the skin of a human subject in need thereof.

25-27. (canceled)
